(19) United States
(12) Patent Application Publication
(10) Pub. No.: US 2010/0316954 A1
(43) Pub. Date: Dec. 16, 2010

(54) MONOMER HAVING LACTONE SKELETON, POLYMER COMPOUND AND PHOTORESIST COMPOSITION

(76) Inventors: Hiroshi Koyama, Hyogo (JP); Mari Sumida, Hyogo (JP); Hiroki Tanaka, Hyogo (JP)

Correspondence Address: BIRCH STEWART KOLASCH & BIRCH, PO BOX 747, FALLS CHURCH, VA, 22040-0747, US

(21) Appl. No.: 12/918,693

(22) PCT Filed: Jan. 21, 2009
(86) PCT No.: PCT/JP2009/000190

§ 371 Date: Aug. 20, 2010

(30) Foreign Application Priority Data
Mar. 12, 2008 (JP) 2008-062550

(51) Int. Cl. G03F 7/20; G03F 7/004; C07D 307/20; C08F 24/00

(52) U.S. Cl. 430/270.1; 430/319; 549/318; 526/270

(57) ABSTRACT

Disclosed is a novel monomer having a lactone skeleton, which is useful typically as a monomer component typically for a highly functional polymer, because, when the monomer is applied typically to a resist resin, the resin is satisfactory stable and resistant typically to chemicals, is highly soluble in organic solvents, and has improved hydrolyzability and/or water solubility after hydrolysis.

The monomer having a lactone skeleton is represented by following Formula (1),

wherein R^{a} represents a hydrogen atom, halogen atom, or substituted or unsubstituted alkyl group having 1 to 6 carbon atoms; R¹ represents a group having a lactone skeleton; and Y represents a bivalent organic group having 1 to 6 carbon atoms.

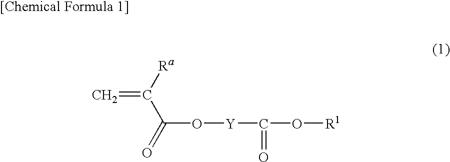

### TECHNICAL FIELD

The present invention relates to monomers, polymer compounds, and photoresist compositions for use in photoresists adopted typically to fine patterning of semiconductor devices (semiconductors); and to processes for manufacturing semiconductor devices using the photoresist compositions.

### BACKGROUND ART

Recent dramatic innovation on lithography patterning techniques in the manufacture of semiconductor devices has made lithographic line widths finer and finer. In lithographic exposure, i-ray and g-ray were initially used to give patterns with broad line widths, and the fabricated semiconductor devices thereby had low capacities. However, recent technological development has allowed the use of KrF excimer laser to give patterns with dramatically finer line widths. Thereafter the technological development has continued so as to adopt ArF excimer laser having a further shorter wavelength to lithographic exposure, and this has been achieved in very recent years. Common resins, i.e., novolak or styrenic resins, have been used in exposure to KrF excimer laser beams. However, in exposure to ArF excimer laser beams, the novolak or styrenic resins have been replaced with resins containing no aromatic moieties, i.e., with alicyclic resins, because the ArF excimer laser beams have a further shorter wavelength of 193 nm, and resins containing aromatic moieties, such as the novolak or styrenic resins, absorb the light of this wavelength. Predominant resins used in exposure to ArF excimer laser beams are acrylic resins. In a mechanism applied in these acrylic resins, acrylic acid has been protected by a protecting group, and upon exposure (light irradiation), an acid is generated and acts to allow the protecting group to leave, i.e., to deprotect the protected acrylic acid into carboxylic acid, and this makes the resins to be soluble in an alkali. Most of currently used protecting groups are alicyclic groups having no polar group. However, polymers derived from monomers containing any of these groups alone show insufficient adhesion to a substrate and lack affinity typically for an alkaline developer, and many acrylic monomers having a polar-group-containing alicyclic skeleton as an ester group have been proposed. Among them, monomers having an alicyclic skeleton containing a lactone ring as a polar group have been highly evaluated on their functions and have been used in large numbers. A part of such monomers can be found in Patent Document 1. Independently, the use of a lactone ring as a monocyclic ester group acting as the protecting group has been proposed typically in Patent Document 2. However, known monomers having such a monocyclic ester group lack functions necessary for resists and seems to be not so widely used. Strong demands are now made on monomers having satisfactory etching resistance, because there is developed a technique called immersion lithography in which a space between a substrate and an exposure system is filled with a liquid having a high density; and this technique allows resist patterns to be finer and finer, and along with this, the resist films tend to have smaller and smaller thicknesses. In addition, strong demands are also made on resins for use in resists to have improved solubility in organic solvents, because resins containing a large quantity of an alicyclic acrylic ester having a lactone ring are insufficient in solubility in organic solvents such as resist solvents.

Patent Document 1: Japanese Unexamined Patent Application Publication (JP-A) No. 2000-026446

Patent Document 2: Japanese Unexamined Patent Application Publication (JP-A) No. H10 (1998)-274852

### DISCLOSURE OF INVENTION

### Problems to be Solved by the Invention

Accordingly, an object of the present invention is to provide a novel monomer having a lactone skeleton, a resin derived from the monomer, a photoresist composition using the resin, and a process for manufacturing a semiconductor device using the photoresist composition, in which the monomer is useful typically as a monomer component typically for a highly functional polymer, because, when the monomer is applied typically to a resist resin, the resin is satisfactorily stable and resistant typically to chemicals, is satisfactorily soluble in organic solvents, and is more satisfactorily hydrolyzable and/or the hydrolyzed product thereof is more satisfactorily soluble in water. Another object of the present invention is to provide a resin which shows high etching resistance when used as a photoresist resin to thereby provide a photoresist resin and a composition containing the photoresist resin for use particularly in immersion lithography.

### Means for Solving the Problems

After intensive investigations on monomers having lactone skeletons for use in photoresist resins, the present inventors have found a monomer which gives a resin that is satisfactorily soluble in solvents and shows high resist performance. The present invention has been made based on these findings.

Specifically, the present invention provides, in an embodiment, a monomer having a lactone skeleton represented by following Formula (1):

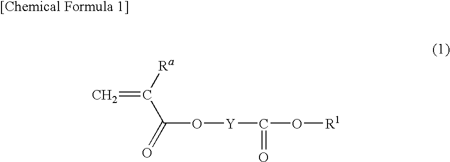

wherein R^{a} represents a hydrogen atom, a halogen atom, or a substituted or unsubstituted alkyl group having 1 to 6 carbon atoms; R¹ represents a group having a lactone skeleton; and Y represents a bivalent organic group having 1 to 6 carbon atoms.

The present invention provides, in another embodiment, a polymer compound including at least a monomeric unit represented by following Formula (1):

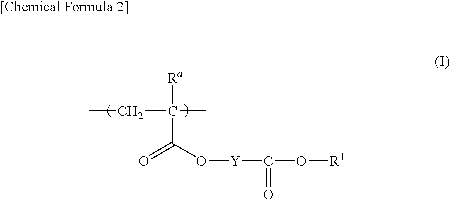

wherein R^{a} represents a hydrogen atom, a halogen atom, or a substituted or unsubstituted alkyl group having 1 to 6 carbon atoms; R¹ represents a group having a lactone skeleton; and Y represents a bivalent organic group having 1 to 6 carbon atoms.

This polymer compound may further include at least a monomeric unit part of which will leave with an acid to allow the residual monomeric unit to be soluble in an alkali, in addition to the monomeric unit represented by Formula (1).

The monomeric unit part of which will leave with an acid to allow the residual monomeric unit to be soluble in an alkali may for example be at least one selected from monomeric units represented by following Formulae (IIa), (IIb), (IIc), and (IId):

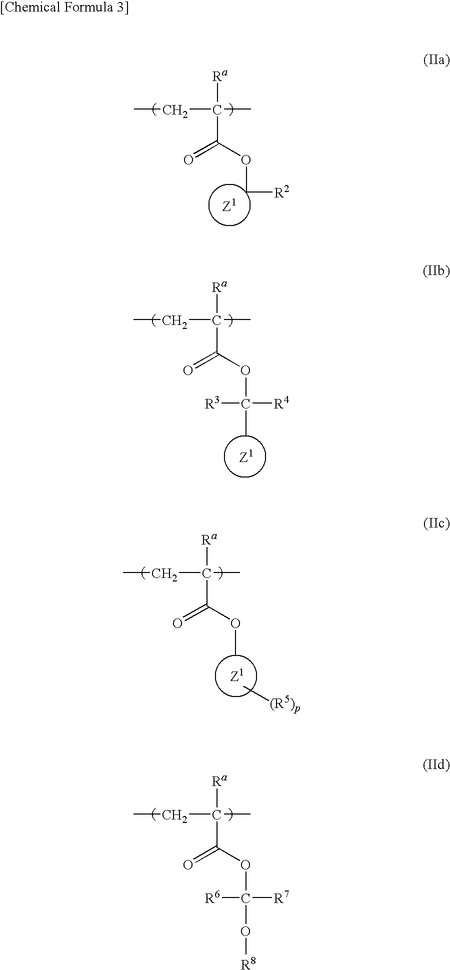

wherein Ring Z¹ represents a substituted or unsubstituted alicyclic hydrocarbon ring having 5 to 20 carbon atoms; R^{a} represents a hydrogen atom, a halogen atom, or a substituted or unsubstituted alkyl group having 1 to 6 carbon atoms; R², R³, and R⁴ are the same as or different from one another and each represent a substituted or unsubstituted alkyl group having 1 to 6 carbon atoms; R⁵s are substituents bound to Ring Z¹, are the same as or different from each other, and each represent an oxo group, an alkyl group, a protected or unprotected hydroxyl group, a protected or unprotected hydroxyalkyl group, or a protected or unprotected carboxyl group, wherein at least one of pR⁵s represents a —COOR^{c} group, and wherein R^{c} represents a substituted or unsubstituted tertiary hydrocarbon group, a tetrahydrofuranyl group, a tetrahydropyranyl group, or an oxepanyl group; “p” denotes an integer of 1 to 3; R⁶ and R⁷ are the same as or different from each other and each represent a hydrogen atom or a substituted or unsubstituted alkyl group having 1 to 6 carbon atoms; and R⁸ represents a hydrogen atom or an organic group, wherein at least two of R⁶, R⁷, and R⁸ may be bound to each other to form a ring with an adjacent atom or atoms.

The polymer compound may further contain at least a monomeric unit containing an alicyclic skeleton having at least one substituent, in addition to the monomeric unit represented by Formula (I).

The monomeric unit containing an alicyclic skeleton having at least one substituent may for example be at least one selected from monomeric units represented by following Formula (III):

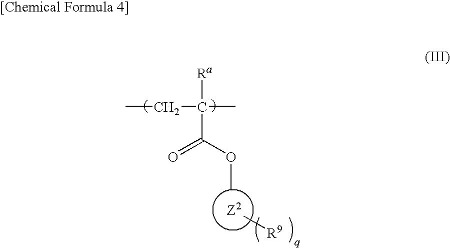

wherein Ring Z² represents an alicyclic hydrocarbon ring having 6 to 20 carbon atoms; R^{a} represents a hydrogen atom, a halogen atom, or a substituted or unsubstituted alkyl group having 1 to 6 carbon atoms; R⁹s are substituents bound to Ring Z², are the same as or different from each other, and each represent an oxo group, an alkyl group, a haloalkyl group, a halogen atom, a protected or unprotected hydroxyl group, a protected or unprotected hydroxyalkyl group, a protected or unprotected mercapto group, a protected or unprotected carboxyl group, a protected or unprotected amino group, or a protected or unprotected sulfonic group; and “q” is the number of R⁹s and denotes an integer of 1 to 5.

The polymer compound preferably contains at least the monomeric unit represented by Formula (I), the monomeric unit part of which will leave with an acid to allow the residual monomeric unit to be soluble in an alkali, and a monomeric unit containing an alicyclic skeleton having at least one substituent selected from hydroxyl groups and hydroxymethyl groups.

The polymer compound may further contain, in addition to the monomeric unit represented by Formula (I), at least another monomeric unit having a lactone skeleton than the monomeric unit represented by Formula (1).

The present invention provides, in still another embodiment, a photoresist composition containing at least the polymer compound and a light-activatable acid generator.

In yet another embodiment, the present invention provides a process for manufacturing a semiconductor device, the process including the step of forming a pattern through the use of the photoresist composition.

### ADVANTAGES

The present invention provides a novel monomer having an ester group containing a lactone skeleton, a resin derived from the monomer, a photoresist composition containing the resin, and a process for manufacturing a semiconductor device using the photoresist composition, in which the monomer is useful as a monomer component of a highly functional polymer compound, because, when the monomer is derived into a polymer compound, the polymer compound is satisfactorily stable and resistant typically to chemicals, is satisfactorily soluble in organic solvents, and can be more satisfactorily hydrolyzable in its ring and/or the hydrolyzed product thereof is more satisfactorily soluble in water. The photoresist composition according to the present invention gives a polymer compound showing improved solubility in an alkaline developer and thereby enables shaper patterning in the manufacture of semiconductor devices.

### BEST MODES FOR CARRYING OUT THE INVENTION

### Monomers Having Lactone Skeleton

Monomers according to the present invention, each having a lactone skeleton, are represented by Formula (1). In Formula (1), R^{a} represents a hydrogen atom, a halogen atom, or a substituted or unsubstituted alkyl group having 1 to 6 carbon atoms; R¹ represents a group having a lactone skeleton; and Y represents a bivalent organic group having 1 to 6 carbon atoms.

As R^{a} in Formula (1), exemplary halogen atoms include fluorine, chlorine, and bromine atoms. Exemplary alkyl groups having 1 to 6 carbon atoms include methyl, ethyl, propyl, isopropyl, butyl, isobutyl, s-butyl, t-butyl, pentyl, and hexyl groups, of which alkyl groups having 1 to 4 carbon atoms are preferred, and methyl group is more preferred. Exemplary substituted alkyl groups having 1 to 6 carbon atoms include chloroalkyl groups such as chloromethyl group; and halo-substituted alkyl groups (haloalkyl groups) having 1 to 6 carbon atoms including fluoroalkyl groups (of which fluoroalkyl groups having 1 to 3 carbon atoms are preferred), such as trifluoromethyl, 2,2,2-trifluoroethyl, and pentafluoroethyl groups.

Preferred as R^{a} are hydrogen atom, methyl group and other alkyl groups having 1 to 3 carbon atoms, and trifluoromethyl group and other haloalkyl groups having 1 to 3 carbon atoms; of which hydrogen atom or methyl group is more preferred.

Exemplary groups having a lactone skeleton as R¹ include groups having a monocyclic lactone skeleton composed of a monocyclic lactone ring such as γ-butyrolactone ring, δ-valerolactonering, or ε-caprolactone ring; and polycyclic lactone skeletons each containing a polycyclic lactone ring such as 6-oxabicyclo[3.2.1^{1,5}]octan-7-one ring or 3-oxatricyclo[4.2.1.0^{4,8}]nonan-2-one ring. Among them, groups having a monocyclic lactone skeleton containing a monocyclic lactone ring are preferred, of which groups having a monocyclic lactone skeleton containing γ-butyrolactone ring are more preferred.

The lactone skeleton may have a substituent. Exemplary substituents include methyl group and other alkyl groups (e.g., alkyl groups having 1 to 4 carbon atoms); trifluoromethyl group and other haloalkyl groups (e.g., haloalkyl groups having 1 to 4 carbon atoms); chlorine atom, fluorine atom, and other halogen atoms; protected or unprotected hydroxyl groups; protected or unprotected hydroxyalkyl groups; protected or unprotected mercapto groups; protected or unprotected carboxyl groups; protected or unprotected amino groups; and protected or unprotected sulfonic groups. Protecting groups commonly used in organic syntheses can be used as protecting groups for the above-mentioned groups. One or more substituents may be substituted on the lactone ring. In an embodiment, the lactone skeleton has two substituents bound to one carbon atom constituting the lactone ring. In this embodiment, the two substituent may be bound to each other to form a cycloalkylidene group (e.g., cyclopentylidene group or cyclohexylidene group) with the carbon atom.

In Formula (1), the lactone skeleton in R¹ may be bound directly, or indirectly with the interposition of a linkage group, to the ester bond (—COO—) shown in the formula. Exemplary linkage groups include alkylene groups (e.g., alkylene groups having 1 to 6 carbon atoms), such as methylene, ethylene, propylene, trimethylene, tetramethylene, and hexamethylene groups.

Representative examples of R¹ include γ-butyrolactone-2-yl groups which may be substituted with one or more alkyl groups having 1 to 4 carbon atoms, such as γ-butyrolactone-2-yl group, 3-methyl-γ-butyrolactone-2-yl group, 3,3-dimethyl-γ-butyrolactone-2-yl group, 4-methyl-γ-butyrolactone-2-yl group, 4,4-dimethyl-γ-butyrolactone-2-yl group, 3,4,4-trimethyl-γ-butyrolactone-2-yl group, 3,3,4-trimethyl-γ-butyrolactone-2-yl group, and 3,3,4,4-tetramethyl-γ-butyrolactone-2-yl group; δ-valerolactone-2-yl groups which may be substituted with one or more alkyl groups having 1 to 4 carbon atoms, such as δ-valerolactone-2-yl group, 3-methyl-δ-valerolactone-2-yl group, 3,3-dimethyl-δ-valerolactone-2-yl group, 4-methyl-5-valerolactone-2-yl group, 4,4-dimethyl-δ-valerolactone-2-yl group, 5-methyl-δ-valerolactone-2-yl group, and 5,5-dimethyl-δ-valerolactone-2-yl group; ε-caprolactone-2-yl groups which may be substituted with one or more alkyl groups having 1 to 4 carbon atoms, such as ε-caprolactone-2-yl group, 2-methyl-ε-caprolactone-2-yl group, and 2,2-dimethyl-ε-caprolactone-2-yl group. Among them, preferred are γ-butyrolactone-2-yl groups each substituted with one or more (especially preferably two) alkyl groups having 1 to 4 carbon atoms, δ-valerolactone-2-yl groups each substituted with one or more (especially preferably two) alkyl groups having 1 to 4 carbon atoms, and ε-caprolactone-2-yl groups each substituted with one or more (especially preferably two) alkyl groups having 1 to 4 carbon atoms, of which 3,3-dimethyl-γ-butyrolactone-2-yl group and other γ-butyrolactone-2-yl groups each substituted with one or more (especially preferably two) alkyl groups having 1 to 4 carbon atoms are especially preferred.

Y represents a bivalent organic group having 1 to 6 carbon atoms. Exemplary bivalent organic groups include methylene, ethylene, propylene, butylene, and other alkylene groups, of which alkylene groups having 1 to 6 carbon atoms are preferred; vinylene and other alkenylene groups, of which alkenylene groups having 2 to 6 carbon atoms are preferred; cycloalkylene groups such as cyclopentylene and cyclohexylene groups; and bivalent organic groups each composed of two or more of these groups bound to each other through a linkage group such as ether bond (—O—), thioether bond (—S—), or ester bond (—COO—; —COO—). Among them, preferred are methylene, ethylene, propylene, and groups each containing both an alkylene group having 1 to 3 carbon atoms and an alkylene group having 1 or 2 carbon atoms bound to each other through ester bond. The listed groups may be substituted with one or more halogen atoms (of which one or more fluorine atoms are preferred), and such halo-substituted groups are also useful herein.

As the monomers having a lactone skeleton, represented by Formula (1), preferred are compounds having a monocyclic lactone skeleton and having a structure in which the lactone ring does not form a polycyclic ring such as adamantane ring, norbornane ring, or norbornene ring. Some of monomers having a lactone ring forming a polycyclic ring may show insufficient hydrolyzability (ring-opening ability), and polymer compounds obtained therefrom containing the lactone ring may not be sufficiently soluble in water after hydrolysis.

Of the monomers having a lactone skeleton, represented by Formula (1), preferred are those having a structure in which a CH₂═C(R^{a})—COOP—COO— group (hereinafter also referred to as an “acyloxy group having a polymerizable unsaturated group”) is bound directly to the lactone ring. When the lactone skeleton in R¹ is a lactone-ring-containing polycyclic lactone skeleton, preferred are monomers having a structure in which the acyloxy group having a polymerizable unsaturated group is bound directly to a carbon atom constituting the lactone ring (e.g., a 5-membered lactone ring), and more preferably those having a structure in which the acyloxy group having a polymerizable unsaturated group is bound directly to a carbon atom at the alpha position of carbonyl group. Such compounds having the acyloxy group having a polymerizable unsaturated group bound directly to the lactone ring show excellent hydrolyzability (ring-opening ability) of the lactone ring, and polymer compounds obtained therefrom having the lactone ring show satisfactory water solubility after hydrolysis. These are probably because of the electron-withdrawing ability of the acyloxy group. In contrast, a compound containing the acyloxy group having a polymerizable unsaturated group indirectly bound to the lactone ring through one or more atoms may show insufficient hydrolyzability (ring-opening ability) of the lactone ring, and a polymer compound obtained therefrom containing the lactone ring may show insufficient water solubility after hydrolysis.

Of the monomers having a lactone skeleton, represented by Formula (1), preferred are monomers having a structure in which one or more alkyl groups (e.g., alkyl groups having 1 to 4 carbon atoms) such as methyl, ethyl, and propyl groups are bound directly to the lactone ring. Among them, more preferred are monomers having a structure in which two alkyl groups are bound directly to one carbon atom constituting the lactone ring. In this case, the two alkyl groups may be bound to each other to form, for example, a cycloalkylidene group (e.g., cyclopentylidene group or cyclohexylidene group) with the carbon atom. Such a monomer having one or more (especially preferably two or more) alkyl groups bound to the lactone ring becomes hydrophobic, gives a pattern whose swelling is suppressed during immersion exposure, whereby the resulting pattern is further fine and has less defect due to residual water, because water can be further satisfactorily removed therefrom.

Representative examples of the monomers having a lactone skeleton, represented by Formula (1), include 2-(meth)acryloyloxyacetoxy-3-methyl-γ-butyrolactones [i.e., α-(meth)acryloyloxyacetoxy-β-methyl-γ-butyrolactones], 2-(meth)acryloyloxyacetoxy-3,3-dimethyl-γ-butyrolactones [i.e., α-(meth)acryloyloxyacetoxy-β,β-dimethyl-γ-butyrolactones], 2-(meth)acryloyloxyacetoxy-4-methyl-γ-butyrolactones, 2-(meth)acryloyloxyacetoxy-4,4-dimethyl-γ-butyrolactones, 2-(meth)acryloyloxyacetoxy-3-methyl-δ-valerolactones, 2-(meth)acryloyloxyacetoxy-3,3-dimethyl-δ-valerolactones, 2-(meth) acryloyloxyacetoxy-4-methyl-δ-valerolactones, 2-(meth)acryloyloxyacetoxy-4,4-dimethyl-δ-valerolactones, 2-(meth) acryloyloxyacetoxy-5-methyl-δ-valerolactones, 2-(meth)acryloyloxyacetoxy-5,5-dimethyl-ε-valerolactones, 2-(meth)acryloyloxyacetoxy-3-methyl-ε-caprolactones, 2-(meth)acryloyloxyacetoxy-3,3-dimethyl-ε-caprolactones, and 2-(methacryloyloxy)ethyl tetrahydro-4,4-dimethyl-2-oxo-3-furanyl succinate.

A reaction path of the synthetic preparation of the monomers having a lactone skeleton, represented by Formula (1), is shown in the following scheme:

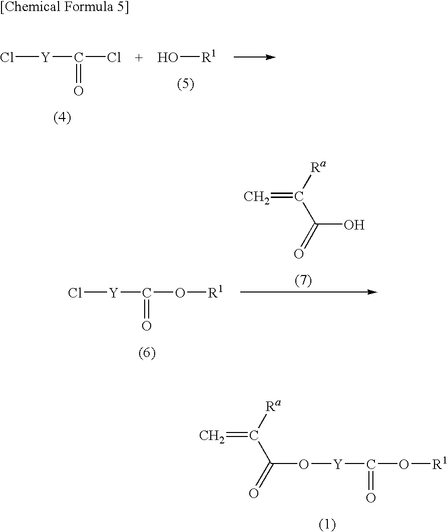

The reaction between an alcohol of Formula (5) having a lactone skeleton and a carboxylic acid chloride of Formula (4) having a group Y substituted by a chlorine atom gives an intermediate of Formula (6). This reaction is preferably performed in the presence of an organic solvent (e.g., acetonitrile). The reaction is also preferably performed in the presence of an organic base. Exemplary organic bases include pyridine; trialkylamines such as dimethylaminopyridine and triethylamine; 1,8-diazabicyclo[5.4.0]undecene-7 (DBU); and tetramethylammonium hydroxide. The reaction may be performed in the presence of a catalyst. Exemplary catalysts include β-zeolite, AMEERLYST, hydrochloric acid, sulfuric acid, p-toluenesulfonic acid, polyphosphoric acid, heteropolyacids (e.g., phosphotungstic acid), boron fluoride, and other acids (including protonic acids and Lewis acids). The reaction temperature is typically about −30° C. to 100° C. The amount of the compound of Formula (4) is typically about 0.8 to 10 moles per 1 mole of the compound of Formula (5).

The obtained intermediate (6) is reacted with an unsaturated carboxylic acid of Formula (7) [e.g., (meth)acrylic acid] into a monomer (1) having a lactone skeleton. This reaction is preferably performed in a solution or suspension using an organic solvent (e.g., N,N-dimethylformamide). The reaction gives hydrogen chloride as a by-product. The reaction is therefore preferably performed in the presence of a base to carry out dehydrochlorination. Exemplary bases include carbonates or hydrogen carbonates of alkali metals, such as potassium carbonate, sodium carbonate, and sodium hydrogen carbonate. It is also desirable to perform the reaction in the presence of a halogen-exchange agent. Exemplary halogen-exchange agents include alkali metal halides such as sodium iodide, potassium iodide, sodium bromide, and potassium bromide. The reaction temperature is typically about −10° C. to 100° C. The amount of the unsaturated carboxylic acid of Formula (7) is typically about 0.8 to 10 moles and preferably about 1 to 2 moles, per 1 mole of the compound of Formula (6).

Each of the intermediate of Formula (6) and the compound of Formula (1) formed as a result of a reaction can be separated and purified through a separation procedure such as filtration, concentration, distillation, extraction, crystallization, recrystallization, or column chromatography, or any combination of these separation procedures.

[Polymer Compounds]

Polymer compounds according to the present invention each contain a monomeric unit represented by Formula (I) which is a monomeric unit (constitutional repeating unit) corresponding to the monomer having a lactone skeleton, represented by Formula (1). The polymer compounds may each contain one or more different monomeric units represented by Formula (I). Such polymer compounds can each be obtained by subjecting any of the monomers having a lactone skeleton, represented by Formula (1), to polymerization.

The monomeric unit represented by Formula (I) helps the resulting polymer to be more soluble in an organic solvent. The lactone ring or ester bond of the monomeric unit is liable to be hydrolyzed to give a polymer having higher water solubility after hydrolysis. Accordingly, the polymer compounds according to the present invention are useful as highly functional polymers adopted typically to applications in which the function of becoming soluble in water through a predetermined treatment is required. The polymer compounds are particularly useful as photoresist resins.

The polymer compounds according to the present invention may further contain one or more other monomeric units in accordance with the intended use and required functions, in addition to the monomeric unit(s) represented by Formula (I). Such other monomeric units can be formed by copolymerizing the monomer(s) having a lactone skeleton, represented by Formula (1), with polymerizable unsaturated monomers corresponding to the other monomeric units.

Examples of the other monomeric units include monomeric units part of which will leave with an acid to allow the polymer compound to be soluble in an alkali, such as monomeric units represented by Formulae (IIa), (IIb), (IIc), and (IId). Polymerizable unsaturated monomers corresponding to the monomeric units represented by Formula (IIa), (IIb), (IIc), and (IId), respectively, are represented by following Formulae (2a), (2b), (2c), and (2d):

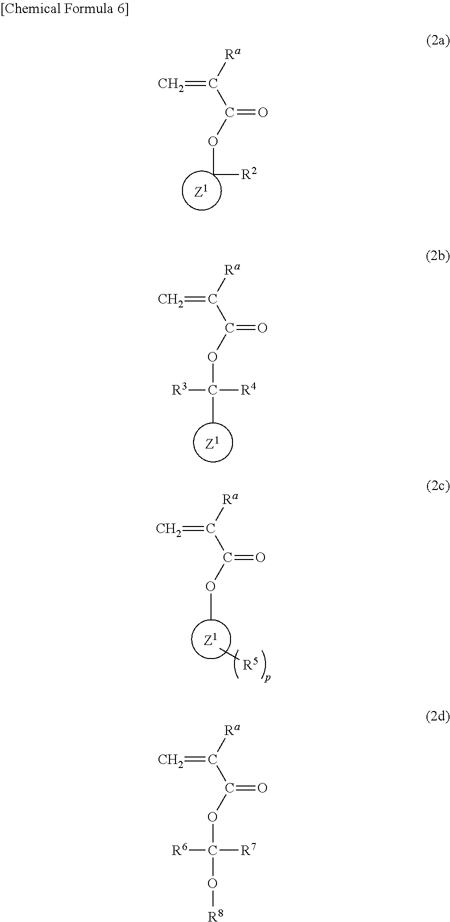

In the formulae, Ring Z¹ represents a substituted or unsubstituted alicyclic hydrocarbon ring having 5 to 20 carbon atoms; R^{a} is as defined above; R², R³, and R⁴ are the same as or different from one another and each represent a substituted or unsubstituted alkyl group having 1 to 6 carbon atoms; R⁵s are substituents bound to Ring Z¹, are the same as or different from each other, and each represent an oxo group, an alkyl group, a protected or unprotected hydroxyl group, a protected or unprotected hydroxyalkyl group, or a protected or unprotected carboxyl group, wherein at least one of pR⁵s represents a —COOR^{c} group, and wherein R^{c} represents a substituted or unsubstituted tertiary hydrocarbon group, a tetrahydrofuranyl group, a tetrahydropyranyl group, or an oxepanyl group. The symbol “p” denotes an integer of 1 to 3. R⁶ and R⁷ are the same as or different from each other and each represent a hydrogen atom or a substituted or unsubstituted alkyl group having 1 to 6 carbon atoms; and R⁸ represents a hydrogen atom or an organic group, in which at least two of R⁶, R⁷, and R⁸ may be bound to each other to form a ring with an adjacent atom or atoms.

In Formulae (2a), (2b), and (2c), the alicyclic hydrocarbon ring having 5 to 20 carbon atoms as Ring Z¹ may be a monocyclic ring, or a polycyclic ring such as fused ring or bridged ring. Representative examples of the alicyclic hydrocarbon ring include cyclohexane ring, cyclooctane ring, cyclodecane ring, adamantane ring, norbornane ring, norbornene ring, bornane ring, isobornane ring, perhydroindene ring, decahydronaphthalene ring, perhydrofluorene ring (tricyclo[7.4.0.0^{3,8}]tridecane ring), perhydroanthracene ring, tricyclo[5.2.1.0^{2,6}]decane ring, tricyclo[4.2.2.1^{2,5}]undecane ring, and tetracyclo[4.4.0.1^{2,5}.1^{7,10}]dodecane ring. The alicyclic hydrocarbon ring may have one or more substituents such as methyl group and other alkyl groups (of which alkyl groups having 1 to 4 carbon atoms are preferred); chlorine atom and other halogen atoms; protected or unprotected hydroxyl groups; oxo groups; and protected or unprotected carboxyl groups. Ring Z¹ is preferably a polycyclic alicyclic hydrocarbon ring (bridged hydrocarbon ring) such as adamantane ring.

Exemplary substituted or unsubstituted alkyl groups having 1 to 6 carbon atoms as R², R³, R⁴, R⁶, and R⁷ in Formulae (2a), (2b), and (2d) include linear or branched chain alkyl groups having 1 to 6 carbon atoms, such as methyl, ethyl, propyl, isopropyl, butyl, isobutyl, s-butyl, t-butyl, and hexyl groups; and haloalkyl groups having 1 to 6 carbon atoms, such as trifluoromethyl group. As R⁵s in Formula (2c), exemplary alkyl groups include linear or branched chain alkyl groups having about 1 to 20 carbon atoms, such as methyl, ethyl, propyl, isopropyl, butyl, isobutyl, s-butyl, t-butyl, hexyl, octyl, decyl, and dodecyl groups. Exemplary protected or unprotected hydroxyl groups as R⁵s include hydroxyl group; and substituted oxy groups including alkoxy groups having 1 to 4 carbon atoms, such as methoxy, ethoxy, and propoxy groups. Exemplary protected or unprotected hydroxyalkyl groups include groups containing any of the above-mentioned protected or unprotected hydroxyl groups bound through an alkylene group having 1 to 6 carbon atoms. Exemplary protected or unprotected carboxyl groups include a —COOR^{d} group. The substituent R^{d} represents a hydrogen atom or an alkyl group, and examples of the alkyl group herein include linear or branched chain alkyl groups having 1 to 6 carbon atoms, such as methyl, ethyl, propyl, isopropyl, butyl, isobutyl, s-butyl, t-butyl, and hexyl groups. Exemplary tertiary hydrocarbon groups as R^{c} in the —COOR^{c} group as R⁵ include t-butyl, t-amyl, 2-methyl-2-adamantyl, and (1-methyl-1-adamantyl)ethyl groups. Exemplary tetrahydrofuranyl groups include 2-tetrahydrofuranyl group; exemplary tetrahydropyranyl groups include 2-tetrahydropyranyl group; and exemplary oxepanyl groups include 2-oxepanyl group.

Exemplary organic groups as R⁸ include groups containing a hydrocarbon group and/or heterocyclic group. Examples of the hydrocarbon group include aliphatic hydrocarbon groups, alicyclic hydrocarbon groups, aromatic hydrocarbon groups, and groups each composed of two or more of these groups bound to each other. Exemplary aliphatic hydrocarbon groups include methyl, ethyl, propyl, isopropyl, butyl, isobutyl, s-butyl, t-butyl, hexyl, octyl, and other linear or branched chain alkyl groups, of which alkyl groups having 1 to 8 carbon atoms are preferred; allyl group and other linear or branched chain alkenyl groups, of which alkenyl groups having 2 to 8 carbon atoms are preferred; and propynyl group and other linear or branched chain alkynyl groups, of which alkynyl groups having 2 to 8 carbon atoms are preferred. Exemplary alicyclic hydrocarbon groups include cyclopropyl, cyclopentyl, cyclohexyl, and other cycloalkyl groups, of which cycloalkyl groups having 3 to 8 members are preferred; cyclopentenyl, cyclohexenyl, and other cycloalkenyl groups, of which cycloalkenyl groups having 3 to 8 members are preferred; adamantyl, norbornyl, and other bridged carbocyclic groups, of which bridged carbocyclic groups having 4 to 20 carbon atoms are preferred. Exemplary aromatic hydrocarbon groups include phenyl, naphthyl, and other aromatic hydrocarbon groups having 6 to 14 carbon atoms. Exemplary groups each composed of an aliphatic hydrocarbon group and an aromatic hydrocarbon group bound to each other include benzyl and 2-phenylethyl groups. Each of these hydrocarbon groups may have one or more substituents. Exemplary substituents include alkyl groups such as alkyl groups having 1 to 4 carbon atoms; haloalkyl groups such as haloalkyl groups having 1 to 4 carbon atoms; halogen atoms; protected or unprotected hydroxyl groups; protected or unprotected hydroxymethyl groups; protected or unprotected carboxyl groups; and oxo group. Protecting groups commonly used in organic syntheses can be used as protecting groups for the above-mentioned groups.

Examples of the heterocyclic group include heterocyclic groups each containing at least one heteroatom selected from oxygen atom, sulfur atom, and nitrogen atom.

Preferred examples of the organic group include alkyl groups having 1 to 8 carbon atoms, and organic groups having a cyclic skeleton. Examples of a “ring” constituting the cyclic skeleton include monocyclic or polycyclic nonaromatic or aromatic carbocycles or heterocyclic rings. Among them, monocyclic or polycyclic nonaromatic carbocycles, and lactone rings are more preferred, in which one or more nonaromatic carbocycles may be fused the lactone rings. Examples of the monocyclic nonaromatic carbocycles include cycloalkane rings having about 3 to 15 members, such as cyclopentane ring and cyclohexane ring.

Examples of the polycyclic nonaromatic carbocycles (bridged carbocycles) include adamantane ring; rings containing a norbornane ring or norbornene ring, such as norbornane ring, norbornene ring, bornane ring, isobornane ring, tricyclo[5.2.1.0^{2,6}]decane ring, and tetracyclo[4.4.0.1^{2,5}.1^{7,10}]dodecane ring; rings derived from polycyclic aromatic fused rings, except for being hydrogenated, such as perhydroindene ring, decahydronaphthalene ring (perhydronaphthalene ring), perhydrofluorene ring (tricyclo[7.4.0.0^{3,8}]tridecane ring), and perhydroanthracene ring, of which fully hydrogenated rings are preferred; and bridged carbocycles each containing, for example, two, three, or four rings, such as tricyclo[4.2.2.1^{2,5}]undecane ring, of which bridged carbocycles having about 6 to 20 carbon atoms are preferred. Examples of the lactone rings include γ-butyrolactone ring, 4-oxatricyclo[4.3.1.1^{3,8}]undecan-5-one ring, 3-oxatricyclo[4.2.1.0^{4,8}]nonan-2-one ring, and 4-oxatricyclo[5.2.1.0^{2,6}]decan-5-one ring.

The ring constituting the cyclic skeleton may have one or more substituents. Exemplary substituents include methyl group and other alkyl groups, of which alkyl groups having 1 to 4 carbon atoms are preferred; trifluoromethyl group and other haloalkyl groups, of which haloalkyl groups having 1 to 4 carbon atoms are preferred; chlorine atom, fluorine atom, and other halogen atoms; protected or unprotected hydroxyl groups; protected or unprotected hydroxyalkyl groups; protected or unprotected mercapto groups; protected or unprotected carboxyl groups; protected or unprotected amino groups; and protected or unprotected sulfonic groups. Protecting groups commonly used in organic syntheses can be used as protecting groups for the above groups.

The ring constituting the cyclic skeleton may be bound to the oxygen atom (oxygen atom at the adjacent position to R⁸) shown in Formula (2d) directly, or indirectly through a linkage group. Examples of the linkage group include linear or branched chain alkylene groups such as methylene, methylmethylene, dimethylmethylene, ethylene, propylene, and trimethylene groups; carbonyl group; oxygen atom (ether bond; —O—); oxycarbonyl group (ester bond; —COO—); aminocarbonyl group (amide bond; —CONH—); and groups each composed of two or more of these.

At least two of R⁶, R⁷, and R⁸ may be bound to each other to form a ring together with an adjacent atom or atoms. Examples of the ring include cycloalkane rings such as cyclopropane ring, cyclopentane ring, and cyclohexane ring; oxygen-containing rings such as tetrahydrofuran ring, tetrahydropyran ring, and oxepane ring; and bridged rings.

There can be stereoisomers in the compounds represented by Formulae (2a), (2b), (2c), and (2d), respectively. Each of such stereoisomers can be used alone or in combination as a mixture.

Representative examples of the compounds represented by Formula (2a) include, but are not limited to, 2-(meth)acryloyloxy-2-methyladamantanes, 1-hydroxy-2-(meth)acryloyloxy-2-methyladamantanes, 5-hydroxy-2-(meth)acryloyloxy-2-methyladamantanes, and 2-(meth)acryloyloxy-2-ethyladamantanes.

Representative examples of the compounds represented by Formula (2b) include, but are not limited to, 1-(1-(meth)acryloyloxy-1-methylethyl)adamantanes, 1-hydroxy-3-(1-(meth)acryloyloxy-1-methylethyl)adamantanes, 1-(1-ethyl-1-(meth)acryloyloxypropyl)adamantanes, 1-(1-(meth)acryloyloxy-1-methylpropyl)adamantanes, and 1-(1-methacryloyloxy-1-methylethyl)cyclohexane.

Representative examples of the compounds represented by Formula (2c) include, but are not limited to, 1-t-butoxycarbonyl-3-(meth)acryloyloxyadamantanes and 1-(2-tetrahydropyranyloxycarbonyl)-3-(meth)acryloyloxyadamantanes.

Representative examples of the compounds represented by Formula (2d) include, but are not limited to, 1-adamantyloxy-1-ethyl (meth)acrylates, 1-adamantylmethyloxy-1-ethyl (meth)acrylates, 2-(1-adamantylethyl)oxy-1-ethyl (meth)acrylates, 1-bornyloxy-1-ethyl (meth)acrylates, 2-norbornyloxy-1-ethyl (meth)acrylates, 2-tetrahydropyranyl (meth)acrylates, and 2-tetrahydrofuranyl (meth)acrylates.

Each of the compounds represented by Formula (2d) can be prepared, for example, by reacting a corresponding vinyl ether compound with (meth)acrylic acid by the catalysis of an acid catalyst according to customary processes. For example, 1-adamantyloxy-1-ethyl (meth)acrylate can be prepared by reacting 1-adamantyl vinyl ether with (meth)acrylic acid in the presence of an acid catalyst.

Examples of the other monomeric units further include, in addition to those mentioned above, monomeric units that can impart or improve properties such as hydrophilicity and/or water solubility of the polymer compounds. Exemplary monomers corresponding to the monomeric units just mentioned above include polar-group-containing monomers such as hydroxyl-containing monomers (including compounds whose hydroxyl group is protected), mercapto-containing monomers (including compounds whose mercapto group is protected), carboxyl-containing monomers (including compounds whose carboxyl group is protected), amino-containing monomers (including compounds whose amino group is protected), sulfonic-containing monomers (including compounds whose sulfonic group is protected), lactone-skeleton-containing monomers, cyclic-ketone-skeleton-containing monomers, acid-anhydride-containing monomers, imide-containing monomers, and other monomers.

Examples of the other monomeric units just mentioned above include monomeric units containing an alicyclic skeleton having at least one substituent, such as monomeric units represented by Formula (III). Polymerizable unsaturated monomers corresponding to the monomeric units represented by Formula (III) are represented by following Formula (3):

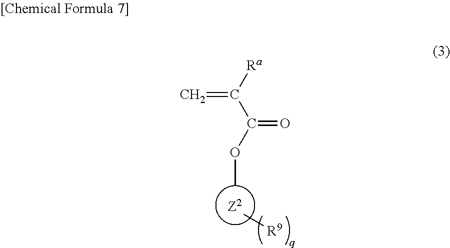

In Formula (3), Ring Z² represents an alicyclic hydrocarbon ring having 6 to 20 carbon atoms; R^{a} is as defined above; R⁹s are substituents bound to Ring Z², are the same as or different from each other, and each represent an oxo group, an alkyl group, a haloalkyl group, a halogen atom, a protected or unprotected hydroxyl group, a protected or unprotected hydroxyalkyl group, a protected or unprotected mercapto group, a protected or unprotected carboxyl group, a protected or unprotected amino group, or a protected or unprotected sulfonic group; and “q” is the number of R⁹s and denotes an integer of 1 to 5.

Of the monomers represented by Formula (3), monomers in which at least one of qR⁹s is an oxo group, a protected or unprotected hydroxyl group, a protected or unprotected hydroxyalkyl group, a protected or unprotected mercapto group, a protected or unprotected carboxyl group, a protected or unprotected amino group, or a protected or unprotected sulfonic group correspond to the polar-group-containing monomers that can impart hydrophilicity and/or water solubility to the polymer or can improve the hydrophilicity and/or water solubility of the polymer.

The alicyclic hydrocarbon ring having 6 to 20 carbon atoms, as Ring Z², may be either a monocyclic ring or a polycyclic ring such as bridged ring. Representative examples of the alicyclic hydrocarbon ring include cyclohexane ring, cyclooctane ring, cyclodecane ring, adamantane ring, norbornane ring, norbornene ring, bornane ring, isobornane ring, perhydroindene ring, decahydronaphthalene ring, perhydrofluorene ring (tricyclo[7.4.0.0^{3,8}]tridecane ring), perhydroanthracene ring, tricyclo[5.2.1.0^{2,6}]decane ring, tricyclo[4.2.2.1^{2,5}]undecane ring, and tetracyclo[4.4.0.1^{2,5}.1^{7,10}]dodecane ring. Of such alicyclic hydrocarbon rings, especially preferred are bridged alicyclic hydrocarbon rings such as adamantane ring.

As R⁹s in Formula (3), exemplary alkyl groups include methyl, ethyl, propyl, isopropyl, butyl, isobutyl, s-butyl, t-butyl, hexyl, octyl, decyl, dodecyl, and other linear or branched chain alkyl groups having about 1 to 20 carbon atoms, of which alkyl groups having 1 to 4 carbon atoms are preferred. Exemplary haloalkyl groups include trifluoromethyl and other haloalkyl groups having about 1 to 20 carbon atoms, of which haloalkyl groups having 1 to 4 carbon atoms are preferred. Exemplary halogen atoms include fluorine atom and chlorine atom. Exemplary protected or unprotected amino groups include amino group; and substituted amino groups including alkyl-substituted amino groups whose alkyl moiety having 1 to 4 carbon atoms, such as methylamino, ethylamino, and propylamino groups.

Exemplary protected or unprotected sulfonic groups include a —SO₃R^{e} group, in which R^{e} represents a hydrogen atom or an alkyl group. Exemplary alkyl groups herein include linear or branched chain alkyl groups having 1 to 6 carbon atoms, such as methyl, ethyl, propyl, isopropyl, butyl, isobutyl, s-butyl, t-butyl, and hexyl groups. The protected or unprotected hydroxyl group, protected or unprotected hydroxyalkyl group, protected or unprotected mercapto group, and protected or unprotected carboxyl group as R⁹s are as above.

Representative examples of the compounds represented by Formula (3) include, but are not limited to, 1-hydroxy-3-(meth) acryloyloxyadamantanes, 1,3-dihydroxy-5-(meth) acryloyloxyadamantanes, 1-carboxy-3-(meth) acryloyloxyadamantanes, 1,3-dicarboxy-5-(meth) acryloyloxyadamantanes, 1-carboxy-3-hydroxy-5-(meth) acryloyloxyadamantanes, 1-t-butoxycarbonyl-3-(meth)acryloyloxyadamantanes, 1,3-bis(t-butoxycarbonyl)-5-(meth)acryloyloxyadamantanes, 1-t-butoxycarbonyl-3-hydroxy-5-(meth)acryloyloxyadamantanes, 1-(2-tetrahydropyranyloxycarbonyl)-3-(meth)acryloyloxyadamantanes, 1,3-bis(2-tetrahydropyranyloxycarbonyl)-5-(meth) acryloyloxyadamantanes, 1-hydroxy-3-(2-tetrahydropyranyloxycarbonyl)-5-(meth)acryloyloxyadamantanes, and 1-(meth)acryloyloxy-4-oxoadamantanes.

Monomers containing an alicyclic skeleton (e.g., adamantane skeleton) having at least one substituent selected from hydroxyl group and hydroxymethyl group are preferred as the monomers corresponding to the monomeric units containing an alicyclic skeleton having at least one substituent.

Examples of the other monomeric units further include monomeric units having a lactone skeleton [other than the monomeric units represented by Formula (I)]. Specific examples of polymerizable unsaturated monomers [i.e., lactone-ring-containing monomers (other than the compounds represented by Formula (1))] corresponding to the monomeric units having a lactone skeleton [other than the monomeric units represented by Formula (I)] include the following compounds.

1-(Meth)acryloyloxy-4-oxatricyclo[4.3.1.1^{3,8}]undecan-5-ones, 1-(meth)acryloyloxy-4,7-dioxatricyclo[4.4.1.1^{3,9}]dodecane-5,8-diones, 1-(meth)acryloyloxy-4,8-dioxatricyclo[4.4.1.1^{3,9}]dodecane-5,7-diones, 1-(meth)acryloyloxy-5,7-dioxatricyclo[4.4.1.1^{3,9}]dodecane-4,8-diones, 5-(meth)acryloyloxy-3-oxatricyclo[4.2.1.0^{4,8}]nonan-2-ones, 5-(meth)acryloyloxy-5-methyl-3-oxatricyclo[4.2.1.0″]nonan-2-ones, 5-(meth)acryloyloxy-6-methyl-3-oxatricyclo[4.2.1.0^{4,8}]nonan-2-ones, 5-(meth)acryloyloxy-9-methyl-3-oxatricyclo[4.2.1.0^{4,8}]nonan-2-ones, 5-(meth)acryloyloxy-9-carboxy-3-oxatricyclo[4.2.1.0^{4,8}]nonan-2-ones, 5-(meth)acryloyloxy-9-methoxycarbonyl-3-oxatricyclo[4.2.1.0^{4,8}]nonan-2-ones, 5-(meth)acryloyloxy-9-ethoxycarbonyl-3-oxatricyclo[4.2.1.0^{4,8}]nonan-2-ones, 5-(meth)acryloyloxy-9-t-butoxycarbonyl-3-oxatricyclo[4.2.1.0^{4,8}]nonan-2-ones, 8-(meth)acryloyloxy-4-oxatricyclo[5.2.1.0^{2,6}]decan-5-ones, 9-(meth)acryloyloxy-4-oxatricyclo[5.2.1.0^{2,6}]decan-5-ones, 4-(meth)acryloyloxy-6-oxabicyclo[3.2.1]octan-7-ones, 4-(meth)acryloyloxy-4-methyl-6-oxabicyclo[3.2.1]octan-7-ones, 4-(meth)acryloyloxy-5-methyl-6-oxabicyclo[3.2.1]octan-7-ones, 4-(meth)acryloyloxy-4,5-dimethyl-6-oxabicyclo[3.2.1]octan-7-ones, 6-(meth)acryloyloxy-2-oxabicyclo[2.2.2]octan-3-ones, 6-(meth)acryloyloxy-6-methyl-2-oxabicyclo[2.2.2]octan-3-ones, 6-(meth)acryloyloxy-1-methyl-2-oxabicyclo[2.2.2]octan-3-ones, 6-(meth)acryloyloxy-1,6-dimethyl-2-oxabicyclo[2.2.2]octan-3-ones, β-(meth)acryloyloxy-γ-butyrolactones, β-(meth)acryloyloxy-α,α-dimethyl-γ-butyrolactones, β-(meth)acryloyloxy-γ,γ-dimethyl-γ-butyrolactones, β-(meth)acryloyloxy-α,α,β-trimethyl-γ-butyrolactones, β-(meth)acryloyloxy-β,γ,γ-trimethyl-γ-butyrolactones, β-(meth)acryloyloxy-α,α,β,γ,γ-pentamethyl-γ-butyrolactones, α-(meth)acryloyloxy-γ-butyrolactones, α-(meth)acryloyloxy-α-methyl-γ-butyrolactones, α-(meth)acryloyloxy-β,β-dimethyl-γ-butyrolactones, α-(meth)acryloyloxy-α,β,β-trimethyl-γ-butyrolactones, α-(meth)acryloyloxy-γ,γ-dimethyl-γ-butyrolactones, α-(meth)acryloyloxy-α,γ,γ-trimethyl-γ-butyrolactones, α-(meth)acryloyloxy-β,β,γ,γ-tetramethyl-γ-butyrolactones, α-(meth)acryloyloxy-α,β,β,γ,γ-pentamethyl-γ-butyrolactones, and γ-(meth)acryloyloxy-γ,γ-dimethyl-γ-butyrolactones.

Examples of the monomeric units having a lactone skeleton [other than the monomeric units represented by Formula (I)] further include monomers each composed of a polycyclic ester group bound directly to (meth)acrylic acid, in which the polycyclic ester group contains both an electron-withdrawing substituent and a lactone skeleton. The monomeric units just mentioned above are represented by following Formula (IV):

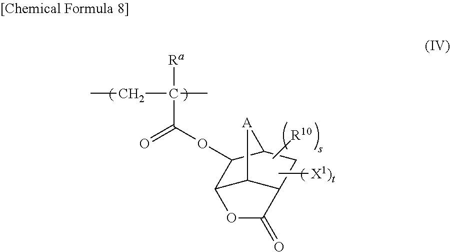

In Formula (IV), R^{a} is as defined above. R¹⁰s are substituents bound to the ring and each represent a halogen atom, an alkyl or haloalkyl group having 1 to 6 carbon atoms, a hydroxyalkyl or hydroxyhaloalkyl group having 1 to 6 carbon atoms whose hydroxyl moiety may be protected by a protecting group, a carboxyl group which may form a salt, or a substituted oxycarbonyl group. “A” represents an alkylene group having 1 to 6 carbon atoms, an oxygen atom, a sulfur atom, or nonbonding. The symbol “s” is the number of R¹⁰s and denotes an integer of 0 to 8. X¹s each represent an electron-withdrawing substituent, and the symbol “t” is the number of X¹s bound to the ring and denotes an integer of 1 to 9. The —COO— group bound to the polymer chain may have either endo or exo configuration.

As R¹⁰s, exemplary halogen atoms include fluorine, chlorine, and bromine atoms. Exemplary alkyl groups having 1 to 6 carbon atoms include methyl, ethyl, propyl, isopropyl, butyl, isobutyl, s-butyl, t-butyl, pentyl, and hexyl groups; of which alkyl groups having 1 to 4 carbon atoms are preferred, and methyl group is more preferred. Exemplary haloalkyl groups (alkyl groups having one or more halogen atoms) having 1 to 6 carbon atoms include chloromethyl group and other chloroalkyl groups; and trifluoromethyl, 2,2,2-trifluoroethyl, pentafluoroethyl, and other fluoroalkyl groups, of which fluoroalkyl groups having 1 to 3 carbon atoms are preferred.

As R¹⁰s, exemplary hydroxyalkyl groups having 1 to 6 carbon atoms include hydroxymethyl, 2-hydroxyethyl, 1-hydroxyethyl, 3-hydroxypropyl, 2-hydroxypropyl, 4-hydroxybutyl, and 6-hydroxyhexyl groups. Exemplary hydroxyhaloalkyl groups having 1 to 6 carbon atoms include difluorohydroxymethyl, 1,1-difluoro-2-hydroxyethyl, 2,2-difluoro-2-hydroxyethyl, and 1,1,2,2-tetrafluoro-2-hydroxyethyl groups. Of such hydroxyalkyl or hydroxyhaloalkyl groups having 1 to 6 carbon atoms, preferred are hydroxyalkyl groups or hydroxyhaloalkyl groups having one or two carbon atoms, of which those having one carbon atoms are more preferred. The protecting group for the hydroxyl group in the hydroxyalkyl or hydroxyhaloalkyl groups having 1 to 6 carbon atoms can be any of protecting groups generally used as hydroxyl-protecting groups in organic syntheses. Examples of such hydroxyl-protecting groups include methyl group, methoxymethyl group, and other groups capable of forming an ether or acetal bond with the oxygen atom constituting the hydroxyl group; and acetyl group, benzoyl group, and other groups capable of forming an ester bond with the oxygen atom constituting the hydroxyl group. Examples of the salt of carboxyl group include alkali metal salts, alkaline earth metal salts, and transition metal salts.

Examples of the substituted oxycarbonyl group include methoxycarbonyl, ethoxycarbonyl, isopropyloxycarbonyl, propoxycarbonyl, and other alkoxycarbonyl groups, of which alkoxy-carbonyl groups whose alkoxy moiety having 1 to 4 carbon atoms are preferred; vinyloxycarbonyl, allyloxycarbonyl, and other alkenyloxycarbonyl groups, of which alkoxy-carbonyl groups whose alkoxy moiety having 2 to 4 carbon atoms are preferred; cyclohexyloxycarbonyl group and other cycloalkyloxycarbonyl groups; and phenyloxycarbonyl group and other aryloxycarbonyl groups.

The symbol “A” represents an alkylene group having 1 to 6 carbon atoms, an oxygen atom, a sulfur atom, or nonbonding. Exemplary alkylene groups having 1 to 6 carbon atoms include alkyl-substituted or -unsubstituted methylene groups, alkyl-substituted or -unsubstituted ethylene groups, and alkyl-substituted or -unsubstituted propylene groups. Among them, an alkylene group having 1 to 6 carbon atoms or nonbonding is preferred as “A”.

Exemplary electron-withdrawing substituents as X¹ include halogen atoms such as fluorine atom; halogenated hydrocarbon groups such as trifluoromethyl group; carboxyl group; alkoxycarbonyl groups such as methoxycarbonyl group; aryloxycarbonyl groups such as phenoxycarbonyl group; acyl groups such as acetyl group; cyano group; aryl groups; 1-alkenyl groups; nitro group; sulfonic acid alkyl ester groups; sulfonic acid; sulfone group; and sulfoxy group. Among them, preferred are fluorine-containing groups such as fluorine atom and trifluoromethyl group; carboxyl group; alkoxycarbonyl groups such as methoxycarbonyl group; acyl groups such as acetyl group; cyano group; and nitro group.

Representative examples of monomers corresponding to the monomeric units represented by Formula (IV) include 1-cyano-5-methacryloyloxy-3-oxatricyclo[4.2.1.0^{4,8}]nonan-2-one, 1-cyano-9-methyl-5-methacryloyloxy-3-oxatricyclo[4.2.1.0^{4,8}]nonan-2-one, 1-cyano-7,7-dimethyl-5-methacryloyloxy-3-oxatricyclo[4.2.1.0^{4,8}]nonan-2-one, 1-cyano-5-methacryloyloxy-3,7-dioxatricyclo[4.2.1.0^{4,8}]nonan-2-one, 1-fluoro-5-methacryloyloxy-3-oxatricyclo[4.2.1.0^{4,8}]nonan-2-one, 1-fluoro-9-methyl-5-methacryloyloxy-3-oxatricyclo[4.2.1.0^{4,8}]nonan-2-one, 1-fluoro-7,7-dimethyl-5-methacryloyloxy-3-oxatricyclo[4.2.1.0^{4,8}]nonan-2-one, 1-fluoro-5-methacryloyloxy-3,7-dioxatricyclo[4.2.1.0^{4,8}]nonan-2-one, 1-trifluoromethyl-5-methacryloyloxy-3-oxatricyclo[4.2.1.0^{4,8}]nonan-2-one, 1-trifluoromethyl-9-methyl-5-methacryloyloxy-3-oxatricyclo[4.2.1.0^{4,8}]nonan-2-one, 1-trifluoromethyl-7,7-dimethyl-5-methacryloyloxy-3-oxatricyclo[4.2.1.0^{4,8}]nonan-2-one, and 1-trifluoromethyl-5-methacryloyloxy-3,7-dioxatricyclo[4.2.1.0^{4,8}]nonan-2-one.

Though not critical, the content of the monomeric unit(s) represented by Formula (I) in the polymer compound according to the present invention is generally about 1 to 90 percent by mole, preferably about 5 to 80 percent by mole, and more preferably about 10 to 60 percent by mole, based on the total amount of monomeric units constituting the polymer. The content of the monomeric unit(s) part of which will leave with an acid to allow the polymer compound to be soluble in an alkali is typically about 10 to 95 percent by mole, preferably about 15 to 90 percent by mole, and more preferably about 20 to 60 percent by mole. The content of the monomeric unit(s) corresponding to at least one monomer selected from the group consisting of hydroxyl-containing monomers, mercapto-containing monomers, and carboxyl-containing monomers is typically about 0 to 60 percent by mole, preferably about 5 to 50 percent by mole, and more preferably about 10 to 40 percent by mole. Examples of the monomeric units just mentioned above include monomeric units represented by Formula (III) in which at least one of qR⁹s is a protected or unprotected hydroxyl group, a protected or unprotected hydroxyalkyl group, a protected or unprotected mercapto group, or a protected or unprotected carboxyl group.

Polymerization of a mixture of monomers for the production of the polymer compounds according to the present invention can be carried out by a technique customarily used in the preparation typically of acrylic polymers, such as solution polymerization, bulk polymerization, suspension polymerization, bulk-suspension polymerization, or emulsion polymerization. Among them, solution polymerization techniques are preferred, of which dropping polymerization is more preferred. Specifically, the drop polymerization can be performed, for example, by any of the following processes (i), (ii), and (iii). In the process (i), a solution of monomers in an organic solvent, and a solution of a polymerization initiator in the organic solvent are previously prepared respectively, and these solutions are respectively added dropwise to the organic solvent held to a constant temperature. In the process (ii), a mixed solution containing monomers and a polymerization initiator in an organic solvent is prepared and added dropwise to the organic solvent held to a constant temperature. In the process (iii), a solution of monomers in an organic solvent, and a solution of a polymerization initiator in the organic solvent are prepared respectively, and the solution of polymerization initiator is added dropwise to the solution of monomers held to a constant temperature.

The solvent used in the polymerization can be any of known solvents. Examples of such solvents include ethers such as chain ethers (e.g., diethyl ether, and glycol ethers such as propylene glycol monomethyl ether) and cyclic ethers (e.g., tetrahydrofuran and dioxane); esters such as methyl acetate, ethyl acetate, butyl acetate, ethyl lactate, and glycol ether esters (e.g., propylene glycol monomethyl ether acetate); ketones such as acetone, methyl ethyl ketone, methyl isobutyl ketone, and cyclohexanone; amides such as N,N-dimethylacetamide and N,N-dimethylformamide; sulfoxides such as dimethyl sulfoxide; alcohols such as methanol, ethanol, and propanol; hydrocarbons such as aromatic hydrocarbons (e.g., benzene, toluene, and xylenes), aliphatic hydrocarbons (e.g., hexane), and alicyclic hydrocarbons (e.g., cyclohexane); and mixtures of these solvents. The polymerization initiator can be any of known polymerization initiators. The polymerization temperature can be chosen as appropriate within the range typically of about 30° C. to 150° C.

Polymers prepared through polymerization can be purified through precipitation or reprecipitation. A solvent for use in precipitation or reprecipitation may be either an organic solvent or water and may also be a solvent mixture. Exemplary organic solvents for use as the precipitation or reprecipitation solvent include hydrocarbons including aliphatic hydrocarbons (e.g., pentane, hexane, heptane, and octane), alicyclic hydrocarbons (e.g., cyclohexane and methylcyclohexane), and aromatic hydrocarbons (e.g., benzene, toluene, and xylenes); halogenated hydrocarbons such as halogenated aliphatic hydrocarbons (e.g., methylene chloride, chloroform, and carbon tetrachloride) and halogenated aromatic hydrocarbons (e.g., chlorobenzene and dichlorobenzenes); nitro compounds such as nitromethane and nitroethane; nitriles such as acetonitrile and benzonitrile; ethers such as chain ethers (e.g., diethyl ether, diisopropyl ether, and dimethoxyethane) and cyclic ethers (e.g., tetrahydrofuran and dioxane); ketones such as acetone, methyl ethyl ketone, and diisobutyl ketone; esters such as ethyl acetate and butyl acetate; carbonates such as dimethyl carbonate, diethyl carbonate, ethylene carbonate, and propylene carbonate; alcohols such as methanol, ethanol, propanol, isopropyl alcohol, and butanol; carboxylic acids such as acetic acid; and solvent mixtures containing these solvents.

Among them, solvent mixtures containing methanol and water and solvents containing at least a hydrocarbon are preferred as organic solvents for use as the precipitation or reprecipitation solvent, of which solvents containing at least an aliphatic hydrocarbon such as hexane are more preferred. The ratio of the hydrocarbon (e.g., an aliphatic hydrocarbon such as hexane) to another solvent [(the former)/(the latter)] in the solvent containing at least the hydrocarbon is typically about 10/90 to 99/1, preferably about 30/70 to 98/2, and more preferably about 50/50 to 97/3, in terms of volume ratio at 25° C.

The polymer compounds have weight-average molecular weights (Mw) of typically about 1000 to 500000 and preferably about 3000 to 50000 and have molecular weight distributions (Mw/Mn) of typically about 1.5 to 2.5. The symbol Mn indicates a number-average molecular weight, and both Mn and Mw are molecular weights in term of a polystyrene.

The polymer compounds according to the present invention are highly stable and resistant typically to chemicals, are satisfactorily soluble in an organic solvent, are highly hydrolyzable, and hydrolyzed products thereof are satisfactorily soluble in water. Accordingly, they can be used as highly functional polymers in various fields.

Photoresist compositions according to the present invention each contain at least any of the polymer compounds according to the present invention and a light-activatable acid generator and generally further contains a resist solvent. Each of the photoresist compositions can be prepared, for example, by adding the light-activatable acid generator to a solution (solution in the resist solvent) of the polymer compound according to the present invention.

Exemplary light-activatable acid generators usable herein include common or known compounds that efficiently generate an acid upon the irradiation with light (exposure), including diazonium salts; iodonium salts such as diphenyliodo hexafluorophosphate; sulfonium salts such as triphenylsulfonium hexafluoroantimonate, triphenylsulfonium hexafluorophosphate, and triphenylsulfonium methanesulfonate; sulfonic acid esters such as 1-phenyl-1-(4-methylphenyl)sulfonyloxy-1-benzoylmethane, 1,2,3-trisulfonyloxymethylbenzene, 1,3-dinitro-2-(4-phenylsulfonyloxymethyl)benzene, and 1-phenyl-1-(4-methylphenylsulfonyloxymethyl)-1-hydroxy-1-benzoylmethane; oxathiazole derivatives; s-triazine derivatives; disulfone derivatives such as diphenyldisulfone; imide compounds; oxime sulfonates; diazonaphthoquinones; and benzoin tosylates. Each of such light-activatable acid generators can be used alone or in combination.

The amount of light-activatable acid generators can be chosen as appropriate according typically to the strength of an acid generated upon the irradiation with light and the contents of respective constitutional repeating units in the polymer (photoresist resin). Typically, the amount can be chosen within the ranges of typically about 0.1 to 30 parts by weight, preferably about 1 to 25 parts by weight, and more preferably about 2 to 20 parts by weight, per 100 parts by weight of the polymer.

Exemplary resist solvents include the glycol solvents, ester solvents, and ketone solvents exemplified as the polymerization solvent, and mixtures of these solvents. Among them, propylene glycol monomethyl ether, propylene glycol monomethyl ether acetate, ethyl lactate, methyl isobutyl ketone, methyl amyl ketone, and mixtures of these are preferred, of which solvents containing at least propylene glycol monomethyl ether acetate are more preferred, and examples thereof include propylene glycol monomethyl ether acetate alone (as a single solvent); a solvent mixture containing both propylene glycol monomethyl ether acetate and propylene glycol monomethyl ether; and a solvent mixture containing both propylene glycol monomethyl ether acetate and ethyl lactate.

The photoresist compositions have polymer concentrations of typically about 10 to 40 percent by weight. The photoresist compositions may further contain one or more other components including alkali-soluble components such as alkali-soluble resins (e.g., novolak resins, phenol resins, imide resins, and carboxyl-containing resins); and colorants (e.g., dyestuffs).

The photoresist composition thus obtained is usable for highly accurate fine patterning (formation of a fine pattern), by applying the photoresist composition to a base or substrate, drying the applied film, applying light through a predetermined mask to the coat film (resist film) (or further performing post-exposure bake) to form a latent image pattern, and subsequently developing the latent image pattern.

Exemplary materials for the base or substrate include silicon wafers, metals, plastics, glass, and ceramics. The application of the photoresist composition can be performed using a common coating device such as spin coater, dip coater, or roller coater. The thickness of the coat film is typically about 0.1 to 20 μm and preferably about 0.3 to 2 μm.

The light application (exposure) can be performed using rays with various wavelengths, such as ultraviolet rays and X-rays. For semiconductor resists, g-ray, i-ray, and excimer laser beams (e.g., XeCl, KrF, KrCl, ArF, or ArCl laser beams) are used. The exposure energy is typically about 1 to 1000 mJ/cm² and preferably about 10 to 500 mJ/cm².

The light application allows the light-activatable acid generator to generate an acid. This acid acts, for example, on a protecting group (leaving group) to leave immediately and to form a group, such as carboxyl group, that helps the polymer compound to be soluble in an alkali, which protecting group is typically for carboxyl group of a constitutional repeating unit capable of being soluble in an alkali by the action of the acid (constitutional repeating unit having an acid-leaving group) of the photoresist polymer compound. Thus, the subsequent development with water or an alkaline developer gives a predetermined pattern highly accurately.

### EXAMPLES

The present invention will be illustrated in further detail with reference to several working examples below. It should be noted, however, that these examples are never construed to limit the scope of the present invention. The weight-average molecular weights (Mw) and the number-average molecular weights (Mn) of sample polymers are values in terms of standard polystyrene as determined through gel permeation chromatography (GPC) using a refractive index detector (RI) and tetrahydrofuran solvent. The gel permeation chromatography was carried out using three columns “KF-806L” (supplied by Showa Denko K.K.) connected in series under conditions of a column temperature of 40° C., an RI temperature of 40° C., and a tetrahydrofuran flow rate of 0.8 ml/min. In Preparation Example 1, a mixture of D- and L-isomers was used as 2-hydroxy-3,3-dimethyl-γ-butyrolactone (also known as pantolactone).

### Preparation Example 1

According to the following reaction scheme, 2-methacryloyloxyacetoxy-3,3-dimethyl-γ-butyrolactone was prepared.

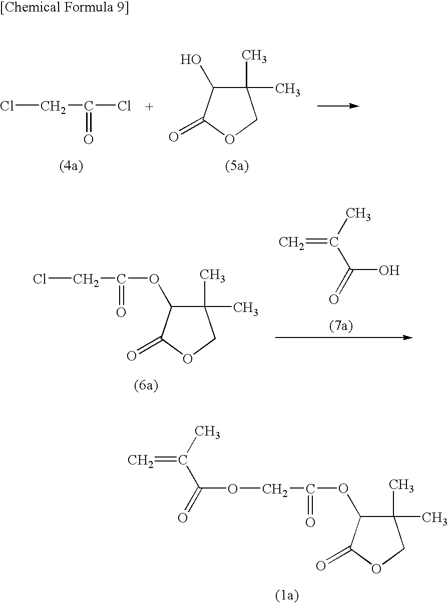

Specifically, 10.0 g (0.0768 mol) of 2-hydroxy-3,3-dimethyl-γ-butyrolactone represented by Formula (5a) and 20.0 g of acetonitrile were placed in a three-neck flask to give a solution, the solution was combined with 38.6 g (0.2536 mol) of 1,8-diazabicyclo[5.4.0]undecene-7 (DBU), followed by heating to an internal temperature of 30° C. Next, in a nitrogen atmosphere, 26.0 g (0.2305 mol) of chloroacetyl chloride represented by Formula (4a) was gradually added dropwise to the solution at an internal temperature of 45° C. or below, followed by stirring at 40° C. for 5 hours. The reaction mixture was then added to and stirred with a mixture of 50 g of ethyl acetate and 50 g of pure water, and an organic layer was isolated from the resulting mixture through separation. The isolated organic layer was sequentially washed with three portions of 36 g of 8 percent by weight aqueous sodium hydrogen carbonate solution, two portions of 36 g of 2 N hydrochloric acid, and three portions of 36 g of 10 percent by weight aqueous sodium chloride solution, concentrated, and thereby yielded 10.8 g (0.0522 mol, 68%) of a crude product of 2-chloroacetoxy-3,3-dimethyl-γ-butyrolactone represented by Formula (6a).

Next, 8.03 g (0.0581 mol) of potassium carbonate, 0.73 g (0.0048 mol) of sodium iodide, 0.008 g of phenothiazine, and 20.00 g of N,N-dimethylformamide were placed in a three-neck flask to give a mixture, and the mixture was combined with 10.0 g (0.0484 mol) of the crude product of 2-chloroacetoxy-3,3-dimethyl-γ-butyrolactone, followed by heating to an internal temperature of 35° C. In a nitrogen atmosphere, 5.00 g (0.0581 mol) of methacrylic acid represented by Formula (7a) was gradually added dropwise thereto, followed by stirring at 35° C. for 2 hours. Next, the mixture was combined with 60.0 g of ethyl acetate, stirred, filtrated, and thereby yielded a dark brown liquid. This liquid was sequentially washed with one portion of 50.0 g of 4 percent by weight sodium hydrogen carbonate and one portion of 25 g of 8 percent by weight sodium hydrogen carbonate, combined with 0.002 g of p-methoxyphenol, and washed with two portions of 25 g of pure water. Next, the organic layer was concentrated to give a crude product, the crude product was purified through silica gel column chromatography and thereby yielded 6.0 g (0.0234 mol, 48%) of 2-methacryloyloxyacetoxy-3,3-dimethyl-γ-butyrolactone represented by Formula (1a). NMR data of these compounds is shown below.

### [2-Chloroacetoxy-3,3-dimethyl-γ-butyrolactone]

¹H-NMR (CDCl₃) δ: 1.15 (s, 3H), 1.24 (s, 3H), 4.05-4.10 (m, 2H), 4.22 (s, 2H), 5.41 (s, 1H)

### [2-Methacryloyloxyacetoxy-3,3-dimethyl-γ-butyrolactone]

¹H-NMR (CDCl₃) δ: 1.12 (s, 3H), 1.23 (s, 3H), 1.99 (m, 3H), 4.03-4.08 (m, 2H), 4.78-4.87 (m, 2H), 5.41 (s, 1H), 5.68-5.69 (m, 1H), 6.24-6.25 (m, 1H)

### Example 2

### Synthesis of Polymer Compound of Following Structure

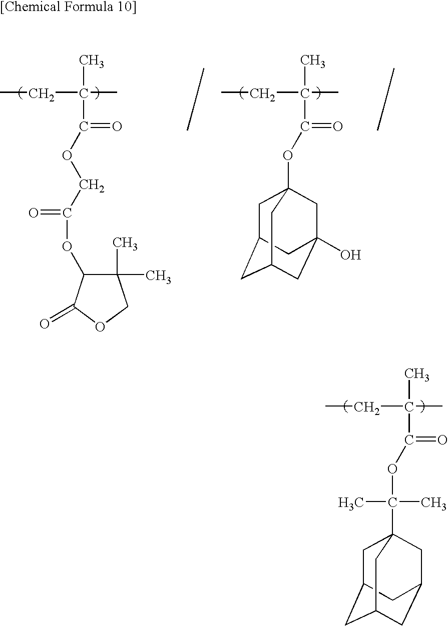

In a nitrogen atmosphere, 41.65 g of propylene glycol monomethyl ether acetate (PGMEA) and 17.85 g of propylene glycol monomethyl ether (PGME) were placed in a round-bottomed flask equipped with a reflux condenser, a stirring bar, and a three-way stopcock to give a mixture; and a monomer solution was added dropwise to the stirred mixture at a constant rate over 6 hours while holding the temperature to 80° C. The monomer solution contained 12.07 g (47.1 mmol) of 2-methacryloyloxyacetoxy-3,3-dimethyl-γ-butyrolactone, 5.57 g (23.5 mmol) of 1-hydroxy-3-methacryloyloxyadamantane, 12.36 g (47.1 mmol) of 1-(1-methacryloyloxy-1-methylethyl)adamantane, 1.80 g of dimethyl 2,2′-azobisisobutylate [trade name “V-601” supplied by Wako Pure Chemical Industries Ltd.], 77.35 g of PGMEA, and 33.15 g of PGME. After the completion of dropwise addition, stirring was continued for further 2 hours. After the completion of polymerization reaction, the resulting reaction mixture was added dropwise to a stirred 9:1 (weight ratio; at 25° C.) mixture of hexane and ethyl acetate in an amount 7 times that of the weight of the reaction mixture, to give precipitates. The precipitates were collected by filtration, dried under reduced pressure, and thereby yielded 27.2 g of the target resin (polymer). The recovered polymer was analyzed through GPC and found to have a weight-average molecular weight (Mw) of 8300 and a molecular weight distribution (Mw/Mn) of 1.92.

### Example 3

### Synthesis of Polymer Compound of Following Structure

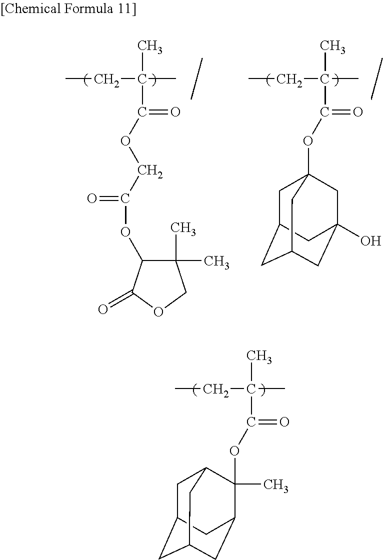

The procedure of Example 2 was repeated, except for using 12.63 g (49.3 mmol) of 2-methacryloyloxyacetoxy-3,3-dimethyl-γ-butyrolactone, 5.82 g (24.6 mmol) of 1-hydroxy-3-methacryloyloxyadamantane, and 11.55 g (49.3 mmol) of 2-methacryloyloxy-2-methyladamantane instead of the monomer components used in Example 2, and thereby yielded 25.2 g of the target resin (polymer). The recovered polymer was analyzed through GPC and found to have a weight-average molecular weight (Mw) of 8900 and a molecular weight distribution (Mw/Mn) of 1.89.

### Example 4

### Synthesis of Polymer Compound of Following Structure

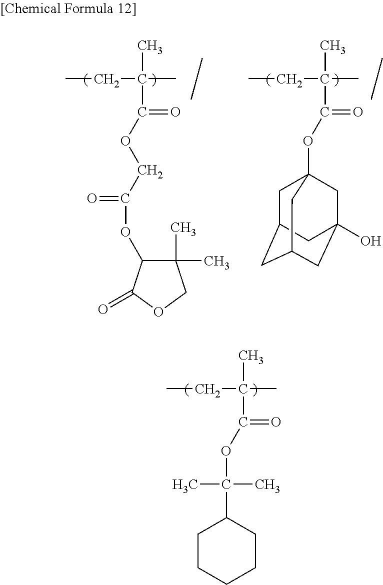

The procedure of Example 2 was repeated, except for using, 13.15 g (51.3 mmol) of 2-methacryloyloxyacetoxy-3,3-dimethyl-γ-butyrolactone, 6.06 g (25.6 mmol) of 1-hydroxy-3-methacryloyloxyadamantane, and 10.79 g (51.3 mmol) of 1-(1-methacryloyloxy-1-methylethyl)cyclohexane instead of the monomer components used in Example 2, and thereby yielded 25.3 g of the target resin (polymer). The recovered polymer was analyzed through GPC and found to have a weight-average molecular weight (Mw) of 9000 and a molecular weight distribution (Mw/Mn) of 1.88.

### Example 5

### Synthesis of Polymer Compound of Following Structure

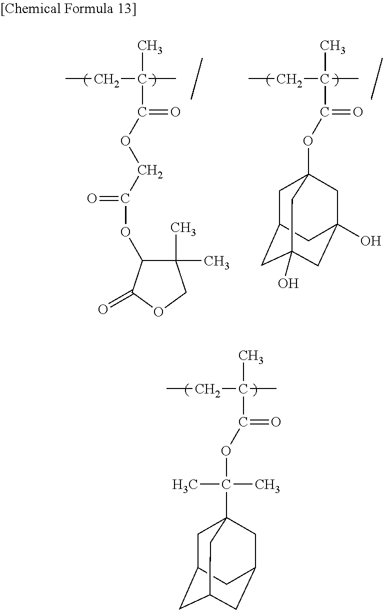

The procedure of Example 2 was repeated, except for using 11.92 g (46.5 mmol) of 2-methacryloyloxyacetoxy-3,3-dimethyl-γ-butyrolactone, 5.87 g (23.3 mmol) of 1,3-dihydroxy-5-methacryloyloxyadamantane, and 12.21 g (46.5 mmol) of 1-(1-methacryloyloxy-1-methylethyl)adamantane instead of the monomer components used in Example 2, and thereby yielded 25.9 g of the target resin (polymer). The recovered polymer was analyzed through GPC and found to have a weight-average molecular weight (Mw) of 8400 and a molecular weight distribution (Mw/Mn) of 1.91.

### Example 6

### Synthesis of Polymer Compound of Following Structure

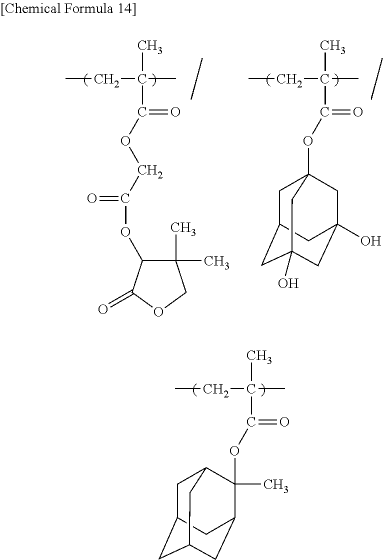

The procedure of Example 2 was repeated, except for using 12.47 g (48.7 mmol) of 2-methacryloyloxyacetoxy-3,3-dimethyl-γ-butyrolactone, 6.14 g (24.3 mmol) of 1,3-dihydroxy-5-methacryloyloxyadamantane, and 11.40 g (48.7 mmol) of 2-methyl-2-methacryloyloxyadamantane instead of the monomer components used in Example 2, and thereby yielded 26.2 g of the target resin (polymer). The recovered polymer was analyzed through GPC and found to have a weight-average molecular weight (Mw) of 8800 and a molecular weight distribution (Mw/Mn) of 1.88.

### Example 7

### Synthesis of Polymer Compound of Following Structure

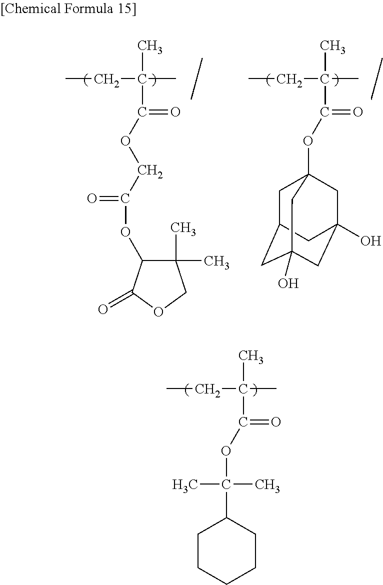

The procedure of Example 2 was repeated, except for using 12.97 g (50.6 mmol) of 2-methacryloyloxyacetoxy-3,3-dimethyl-γ-butyrolactone, 6.39 g (25.3 mmol) of 1,3-dihydroxy-5-methacryloyloxyadamantane, and 10.64 g (50.6 mmol) of 1-(1-methacryloyloxy-1-methylethyl)cyclohexane instead of the monomer components used in Example 2, and thereby yielded 26.1 g of the target resin (polymer). The recovered polymer was analyzed through GPC and found to have a weight-average molecular weight (Mw) of 8900 and a molecular weight distribution (Mw/Mn) of 1.92.

### Comparative Example 1

### Synthesis of Polymer Compound of Following Structure

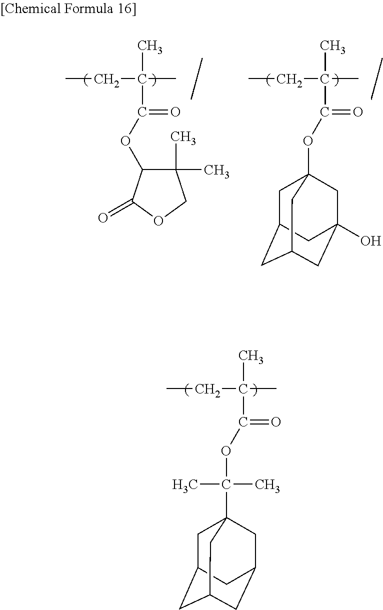

The procedure of Example 2 was repeated, except for using 10.27 g (51.8 mmol) of 2-methacryloyloxy-3,3-dimethyl-γ-butyrolactone, 6.12 g (25.9 mmol) of 1-hydroxy-3-methacryloyloxyadamantane, and 13.60 g (51.8 mmol) of 1-(1-methacryloyloxy-1-methylethyl)adamantane instead of 12.07 g (47.1 mmol) of 2-methacryloyloxyacetoxy-3,3-dimethyl-γ-butyrolactone, 5.57 g (23.5 mmol) of 1-hydroxy-3-methacryloyloxyadamantane, and 12.36 g (47.1 mmol) of 1-(1-methacryloyloxy-1-methylethyl)adamantane used in Example 2, and thereby yielded 26.6 g of the target resin (polymer). The recovered polymer was analyzed through GPC and found to have a weight-average molecular weight (Mw) of 9100 and a molecular weight distribution (Mw/Mn) of 1.89.

### Comparative Example 2

### Synthesis of Polymer Compound of Following Structure

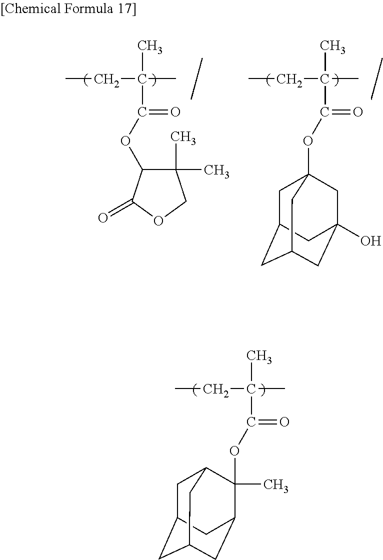

The procedure of Example 3 was repeated, except for using 10.80 g (54.5 mmol) of 2-methacryloyloxy-3,3-dimethyl-γ-butyrolactone, 6.44 g (27.3 mmol) of 1-hydroxy-3-methacryloyloxyadamantane, and 12.76 g (54.5 mmol) of 2-methacryloyloxy-2-methyladamantane instead of 12.63 g (49.3 mmol) of 2-methacryloyloxyacetoxy-3,3-dimethyl-γ-butyrolactone, 5.82 g (24.6 mmol) of 1-hydroxy-3-methacryloyloxyadamantane, and 11.55 g (49.3 mmol) of 2-methacryloyloxy-2-methyladamantane used in Example 3, and thereby yielded 26.3 g of the target resin (polymer). The recovered polymer was analyzed through GPC and found to have a weight-average molecular weight (Mw) of 9200 and a molecular weight distribution (Mw/Mn) of 1.91.

### Comparative Example 3

### Synthesis of Polymer Compound of Following Structure

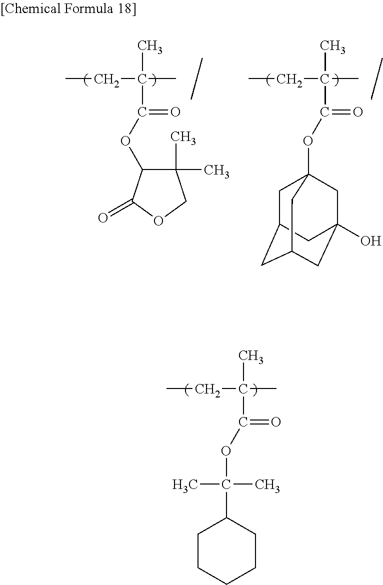

The procedure of Example 4 was repeated, except for using 11.29 g (57.0 mmol) of 2-methacryloyloxy-3,3-dimethyl-γ-butyrolactone, 6.73 g (28.5 mmol) of 1-hydroxy-3-methacryloyloxyadamantane, and 11.98 g (57.0 mmol) of 1-(1-methacryloyloxy-1-methylethyl)cyclohexane instead of 13.15 g (51.3 mmol) of 2-methacryloyloxyacetoxy-3,3-dimethyl-γ-butyrolactone, 6.06 g (25.6 mmol) of 1-hydroxy-3-methacryloyloxyadamantane, and 10.79 g (51.3 mmol) of 1-(1-methacryloyloxy-1-methylethyl)cyclohexane used in Example 4, and thereby yielded 25.1 g of the target resin (polymer). The recovered polymer was analyzed through GPC and found to have a weight-average molecular weight (Mw) of 9700 and a molecular weight distribution (Mw/Mn) of 1.90.

### Comparative Example 4

### Synthesis of Polymer Compound of Following Structure

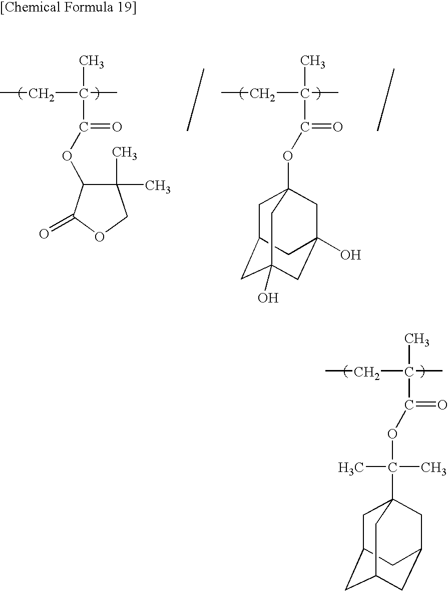

The procedure of Example 5 was repeated, except for using 10.13 g (51.1 mmol) of 2-methacryloyloxy-3,3-dimethyl-γ-butyrolactone, 6.45 g (25.6 mmol) of 1,3-dihydroxy-5-methacryloyloxyadamantane, and 13.42 g (51.1 mmol) of 1-(1-methacryloyloxy-1-methylethyl)adamantane instead of 11.92 g (46.5 mmol) of 2-methacryloyloxyacetoxy-3,3-dimethyl-γ-butyrolactone, 5.87 g (23.3 mmol) of 1,3-dihydroxy-5-methacryloyloxyadamantane, and 12.21 g (46.5 mmol) of 1-(1-methacryloyloxy-1-methylethyl)adamantane used in Example 5, and thereby yielded 27.0 g of the target resin (polymer). The recovered polymer was analyzed through GPC and found to have a weight-average molecular weight (Mw) of 9200 and a molecular weight distribution (Mw/Mn) of 1.90.

### Comparative Example 5

### Synthesis of Polymer Compound of Following Structure

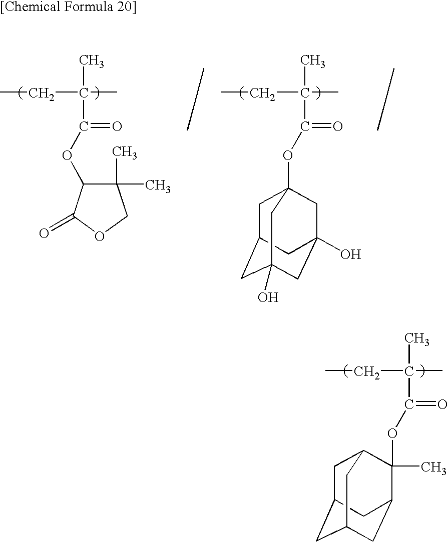

The procedure of Example 6 was repeated, except for using 10.65 g (53.7 mmol) of 2-methacryloyloxy-3,3-dimethyl-γ-butyrolactone, 6.78 g (26.9 mmol) of 1,3-dihydroxy-5-methacryloyloxyadamantane, and 12.58 g (53.7 mmol) of 2-methyl-2-methacryloyloxyadamantane instead of 12.47 g (48.7 mmol) of 2-methacryloyloxyacetoxy-3,3-dimethyl-γ-butyrolactone, 6.14 g (24.3 mmol) of 1,3-dihydroxy-5-methacryloyloxyadamantane, 11.40 g (48.7 mmol) of 2-methyl-2-methacryloyloxyadamantane used in Example 6, and thereby yielded 27.1 g of the target resin (polymer). The recovered polymer was analyzed through GPC and found to have a weight-average molecular weight (Mw) of 9600 and a molecular weight distribution (Mw/Mn) of 1.99.

### Comparative Example 6

### Synthesis of Polymer Compound of Following Structure

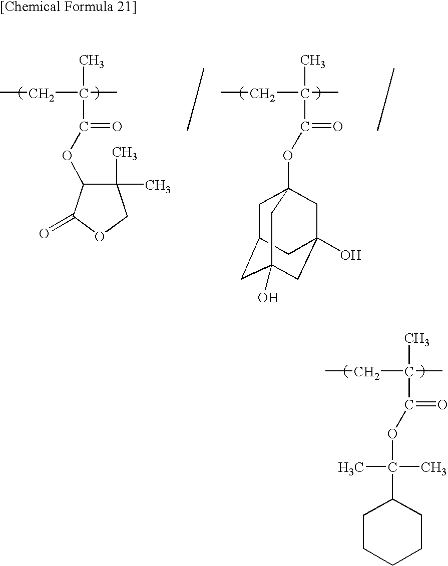

The procedure of Example 7 was repeated, except for using 11.12 g (56.1 mmol) of 2-methacryloyloxy-3,3-dimethyl-γ-butyrolactone, 7.80 g (28.1 mmol) of 1,3-dihydroxy-5-methacryloyloxyadamantane, and 11.80 g (56.1 mmol) of 1-(1-methacryloyloxy-1-methylethyl)cyclohexane instead of 12.97 g (50.6 mmol) of 2-methacryloyloxyacetoxy-3,3-dimethyl-γ-butyrolactone, 6.39 g (25.3 mmol) of 1,3-dihydroxy-5-methacryloyloxyadamantane, and 10.64 g (50.6 mmol) of 1-(1-methacryloyloxy-1-methylethyl)cyclohexane used in Example 7, and thereby yielded 25.8 g of the target resin (polymer).

The recovered polymer was analyzed through GPC and found to have a weight-average molecular weight (Mw) of 9800 and a molecular weight distribution (Mw/Mn) of 1.92.

### Preparation Example 2

According to the following reaction scheme, 2-(methacryloyloxy)ethyl tetrahydro-4,4-dimethyl-2-oxo-3-furanyl succinate was prepared.

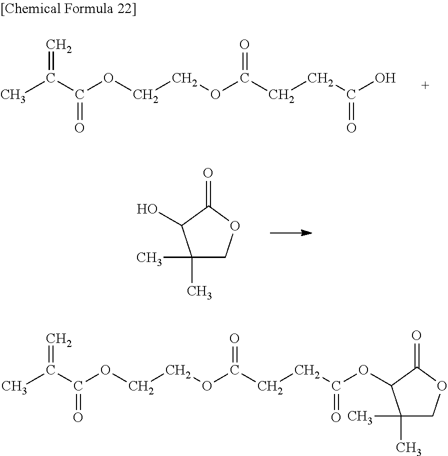

In a nitrogen-purged 500-ml three-neck flask equipped with a stirrer were placed 46.0 g (0.20 mol) of 2-methacryloyloxyethyl hydrogen succinate and 180 g of acetonitrile. After cooling to 5° C., 2.44 g (0.02 mol) of 4-dimethylaminopyridine, 39.3 g (0.205 mol) of 1-ethyl-3-(3-(dimethylaminopropyl)carbodiimide hydrochloride, and 13.0 g (0.10 mol) of pantolactone were added, followed by reacting at a liquid temperature of 25° C. for 7 hours. The reaction mixture was combined with 300 cc of ethyl acetate, sequentially washed with four portions of 300 ml of 10% aqueous sodium carbonate solution, two portions of 300 ml of 2 N hydrochloric acid, and two portions of 300 ml of 10% aqueous sodium chloride solution (brine), followed by concentration under reduced pressure. The concentrated residue was purified through silica gel column chromatography and thereby yielded 26.3 g (0.077 mol, in a yield of 77%) of 2-(methacryloyloxy)ethyl tetrahydro-4,4-dimethyl-2-oxo-3-furanyl succinate. The NMR data of this compound is shown below.

¹H-NMR (CDCl₃) δ: 6.13 (m, 1H), 5.60 (m, 1H), 5.38 (s, 1H), 4.36 (s, 4H), 4.02-4.07 (m, 2H), 2.66-2.82 (m, 4H), 1.95 (s, 3H), 1.21 (s, 3H), 1.12 (s, 3H)

### Example 8

### Synthesis of Polymer Compound of Following Structure

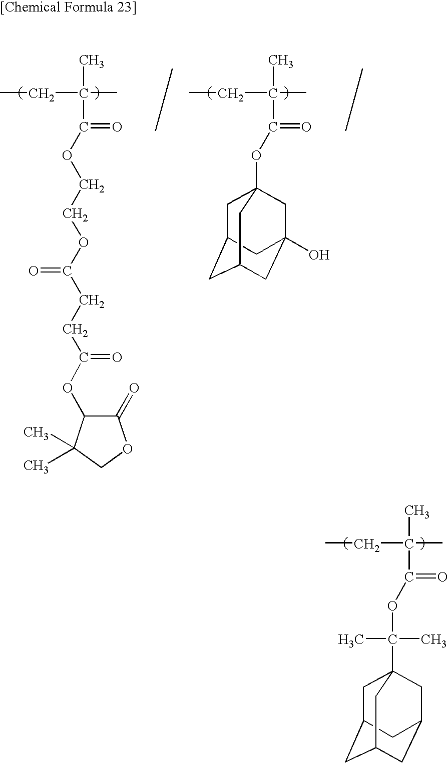

The procedure of Example 2 was repeated, except for using 14.21 g (41.6 mmol) of 2-(methacryloyloxy)ethyl tetrahydro-4,4-dimethyl-2-oxo-3-furanyl succinate, 4.90 g (20.8 mmol) of 1-hydroxy-3-methacryloyloxyadamantane, and 10.89 g (41.6 mmol) of 1-(1-methacryloyloxy-1-methylethyl)adamantane instead of the monomer components used in Example 2, and thereby yielded 27.9 g of the target resin (polymer). The recovered polymer was analyzed through GPC and found to have a weight-average molecular weight (Mw) of 9500 and a molecular weight distribution (Mw/Mn) of 1.91.

### Example 9

### Synthesis of Polymer Compound of Following Structure

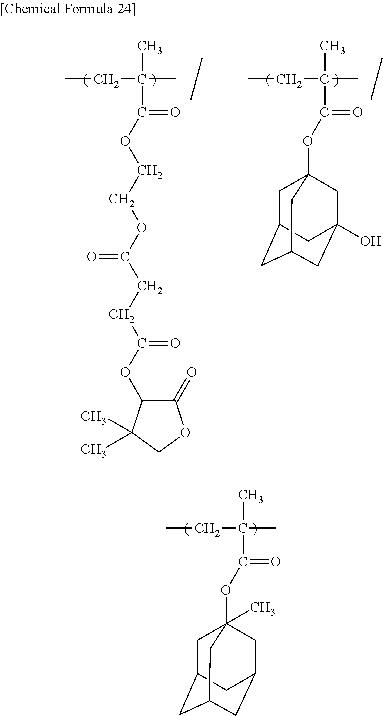

The procedure of Example 2 was repeated, except for using 14.78 g (43.2 mmol) of 2-(methacryloyloxy)ethyl tetrahydro-4,4-dimethyl-2-oxo-3-furanyl succinate, 5.10 g (21.6 mmol) of 1-hydroxy-3-methacryloyloxyadamantane, 10.11 g (43.2 mmol) of 2-methyl-2-methacryloyloxyadamantane instead of the monomer components used in Example 2, and thereby yielded 27.0 g of the target resin (polymer). The recovered polymer was analyzed through GPC and found to have a weight-average molecular weight (Mw) of 9300 and a molecular weight distribution (Mw/Mn) of 1.93.

### Example 10

### Synthesis of Polymer Compound of Following Structure

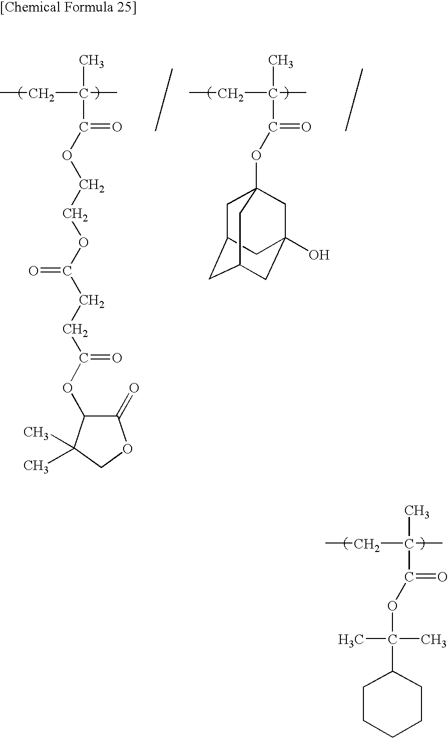

The procedure of Example 2 was repeated, except for using 15.31 g (44.7 mmol) of 2-(methacryloyloxy)ethyl tetrahydro-4,4-dimethyl-2-oxo-3-furanyl succinate, 5.28 g (22.4 mmol) of 1-hydroxy-3-methacryloyloxyadamantane, 9.41 g (44.7 mmol) of 1-(1-methacryloyloxy-1-methylethyl)cyclohexane instead of the monomer components used in Example 2, and thereby yielded 28.5 g of the target resin (polymer). The recovered polymer was analyzed through GPC and found to have a weight-average molecular weight (Mw) of 9500 and a molecular weight distribution (Mw/Mn) of 1.89.

### Example 11

### Synthesis of Polymer Compound of Following Structure

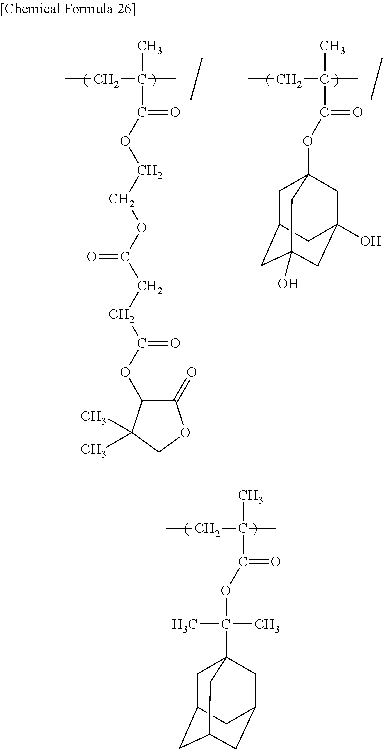

The procedure of Example 2 was repeated, except for using 17.10 g (49.9 mmol) of 2-(methacryloyloxy)ethyl tetrahydro-4,4-dimethyl-2-oxo-3-furanyl succinate, 5.04 g (20.0 mmol) of 1,3-dihydroxy-5-methacryloyloxyadamantane, 7.86 g (30.0 mmol) of 1-(1-methacryloyloxy-1-methylethyl)adamantane instead of the monomer components used in Example 2, and thereby yielded 28.5 g of the target resin (polymer). The recovered polymer was analyzed through GPC and found to have a weight-average molecular weight (Mw) of 9700 and a molecular weight distribution (Mw/Mn) of 1.91.

### Example 12

### Synthesis of Polymer Compound of Following Structure

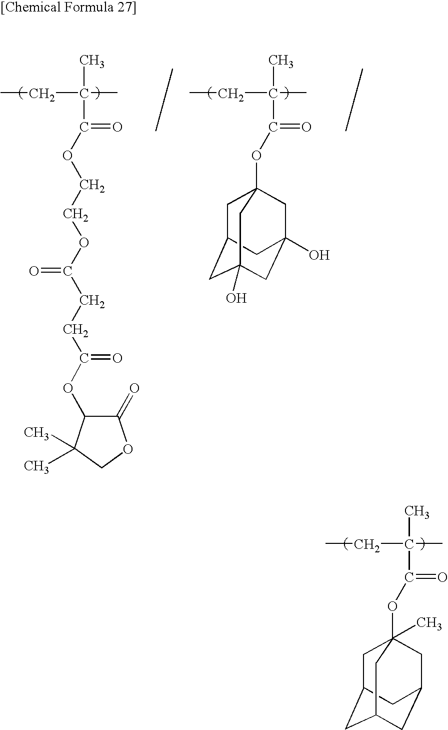

The procedure of Example 2 was repeated, except for using 17.59 g (51.4 mmol) of 2-(methacryloyloxy)ethyl tetrahydro-4,4-dimethyl-2-oxo-3-furanyl succinate, 5.19 g (20.6 mmol) of 1,3-dihydroxy-5-methacryloyloxyadamantane, 7.22 g (30.8 mmol) of 2-methyl-2-methacryloyloxyadamantane instead of the monomer components used in Example 2, and thereby yielded 26.8 g of the target resin (polymer). The recovered polymer was analyzed through GPC and found to have a weight-average molecular weight (Mw) of 8900 and a molecular weight distribution (Mw/Mn) of 1.88.

### Example 13

### Synthesis of Polymer Compound of Following Structure

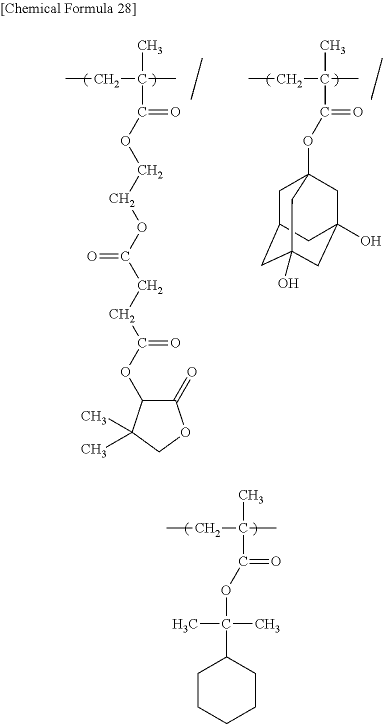

The procedure of Example 2 was repeated, except for using 18.04 g (52.7 mmol) of 2-(methacryloyloxy)ethyl tetrahydro-4,4-dimethyl-2-oxo-3-furanyl succinate, 5.32 g (21.1 mmol) of 1,3-dihydroxy-5-methacryloyloxyadamantane, and 6.65 g (31.6 mmol) of 1-(1-methacryloyloxy-1-methylethyl)cyclohexane instead of the monomer components used in Example 2, and thereby yielded 28.0 g of the target resin (polymer). The recovered polymer was analyzed through GPC and found to have a weight-average molecular weight (Mw) of 9000 and a molecular weight distribution (Mw/Mn) of 1.89.

Evaluation Tests

The photoresist polymer resins prepared in the examples and comparative examples were each combined with and dissolved in propylene glycol monomethyl ether acetate (PGMEA) and propylene glycol monomethyl ether (PGME) so as to give a series of solutions having a polymer concentration of 20 percent by weight in a 6:4 (by weight) mixture of PGMEA and PGME. The polymer resins according to Examples 2 to 7 were immediately dissolved, whereas the polymer resins according to Comparative Examples 1 to 6 required durations 2 to 4 times longer than those for the examples to be dissolved in the solvent mixture. Each of the resulting photoresist polymer solutions was combined with 10 parts by weight of triphenylsulfonium hexafluoroantimonate per 100 parts by weight of the polymer, further combined with PGMEA to give a polymer concentration of 15 percent by weight, filtrated through a filter with a pore size of 0.02 μm, and thereby yielded a series of photoresist compositions. The polymer solutions according to Examples 2 to 7 and 8 to 13 showed good filterability and could be immediately filtered through the filter with a pore size of 0.02 μm, whereas the polymer solutions according to Comparative Examples 1 to 6 required durations about 5 times longer than those for the polymer solutions according to the examples to be filtered through the filter. The filtration speed of the polymer solutions according to the comparative examples became lower particularly in the latter half of the filtration, anticipating frequent filter exchange.

Each of the resulting photoresist compositions was applied to a silicon wafer by spin coating to give a photosensitive layer 0.7 μm thick. The photosensitive layer was prebaked on a hot plate at a temperature of 100° C. for 150 seconds, exposed to ArF excimer laser beams having a wavelength of 193 nm through a mask at an irradiance of 30 mJ/cm², followed by post-baking at a temperature of 100° C. for 60 seconds. Next, the layer was developed with 2.38 M aqueous tetramethylammonium hydroxide solution for 60 seconds and rinsed with ultrapure water. Though both the photoresist polymer solutions according to the examples and the comparative examples gave 0.25-μm line-and-space patterns, the line-and-space patterns obtained from the photoresist polymer solutions according to Examples 2 to 7 and 8 to 13 were apparently clearer than those obtained from the photoresist polymer solutions according to the comparative examples.

1. A monomer having a lactone skeleton represented by following Formula (1): 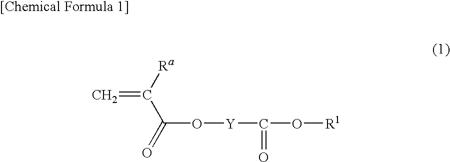
wherein R^{a} represents a hydrogen atom, a halogen atom, or a substituted or unsubstituted alkyl group having 1 to 6 carbon atoms; R¹ represents a group having a lactone skeleton; and Y represents a bivalent organic group having 1 to 6 carbon atoms.

2. A polymer compound comprising at least a monomeric unit represented by following Formula (I): 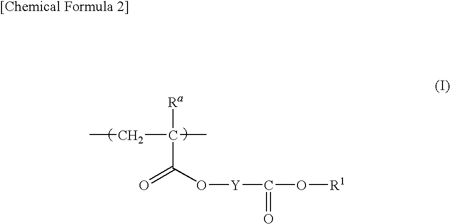
wherein R^{a} represents a hydrogen atom, a halogen atom, or a substituted or unsubstituted alkyl group having 1 to 6 carbon atoms; R¹ represents a group having a lactone skeleton; and Y represents a bivalent organic group having 1 to 6 carbon atoms.

3. The polymer compound according to claim 2, further comprising at least a monomeric unit part of which will leave with an acid to allow the residual monomeric unit to be soluble in an alkali, in addition to the monomeric unit represented by Formula (I).

4. The polymer compound according to claim 3, wherein the monomeric unit part of which will leave with an acid to allow the residual monomeric unit to be soluble in an alkali is at least one selected from the group consisting of monomeric units represented by following Formulae (IIa), (IIb), (IIc), and (IId): 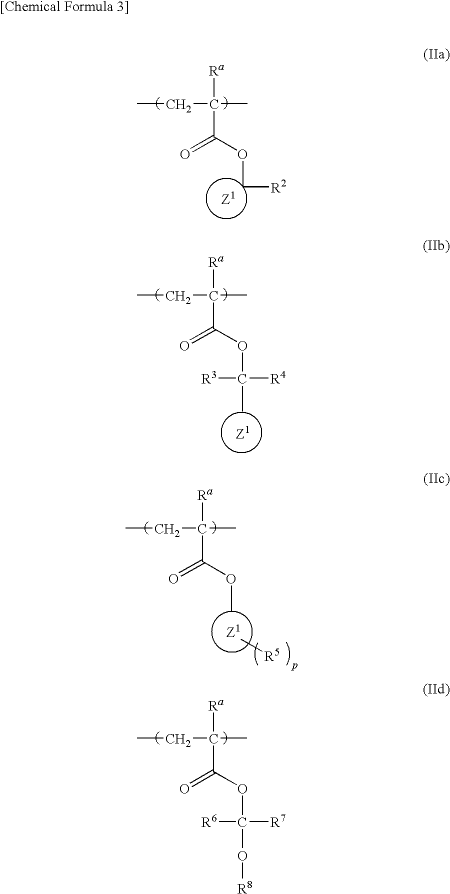
wherein Ring Z¹ represents a substituted or unsubstituted alicyclic hydrocarbon ring having 5 to 20 carbon atoms; R^{a} represents a hydrogen atom, a halogen atom, or a substituted or unsubstituted alkyl group having 1 to 6 carbon atoms; R², R³, and R⁴ are the same as or different from one another and each represent a substituted or unsubstituted alkyl group having 1 to 6 carbon atoms; R⁵s are substituents bound to Ring Z¹, are the same as or different from each other, and each represent an oxo group, an alkyl group, a protected or unprotected hydroxyl group, a protected or unprotected hydroxyalkyl group, or a protected or unprotected carboxyl group, wherein at least one of pR⁵s represents a —COOR^{e} group, and wherein R^{e} represents a substituted or unsubstituted tertiary hydrocarbon group, a tetrahydrofuranyl group, a tetrahydropyranyl group, or an oxepanyl group; “p” denotes an integer of 1 to 3; R⁶ and R⁷ are the same as or different from each other and each represent a hydrogen atom or a substituted or unsubstituted alkyl group having 1 to 6 carbon atoms; and R⁸ represents a hydrogen atom or an organic group, wherein at least two of R⁶, R⁷, and R⁸ may be bound to each other to form a ring with an adjacent atom or atoms.

5. The polymer compound according to any one of claims 2 to 4, further comprising at least a monomeric unit containing an alicyclic skeleton having at least one substituent, in addition to the monomeric unit represented by Formula (I).

6. The polymer compound according to claim 5, wherein the monomeric unit containing an alicyclic skeleton having at least one substituent is at least one selected from the group consisting of monomeric units represented by following Formula (III): 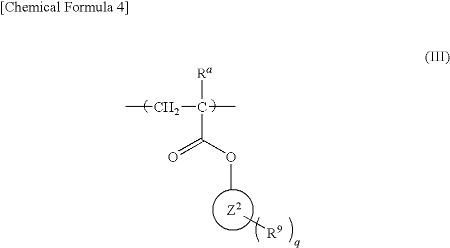
wherein Ring Z² represents an alicyclic hydrocarbon ring having 6 to 20 carbon atoms; R^{a} represents a hydrogen atom, a halogen atom, or a substituted or unsubstituted alkyl group having 1 to 6 carbon atoms; R⁹s are substituents bound to Ring Z², are the same as or different from each other, and each represent an oxo group, an alkyl group, a haloalkyl group, a halogen atom, a protected or unprotected hydroxyl group, a protected or unprotected hydroxyalkyl group, a protected or unprotected mercapto group, a protected or unprotected carboxyl group, a protected or unprotected amino group, or a protected or unprotected sulfonic group; and “q” is the number of R⁹s and denotes an integer of 1 to 5.

7. The polymer compound according to claim 3, comprising the monomeric unit represented by Formula (I); the monomeric unit part of which will leave with an acid to allow the residual monomeric unit to be soluble in an alkali; and a monomeric unit containing an alicyclic skeleton having at least one substituent selected from hydroxyl groups and hydroxymethyl groups.

8. The polymer compound according to claim 2, further comprising, in addition to the monomeric unit represented by Formula (I), another monomeric unit having a lactone skeleton than the monomeric unit represented by Formula (I).

9. A photoresist composition comprising at least the polymer compound according to claim 2; and a light-activatable acid generator.

10. A process for manufacturing a semiconductor device, the process comprising the step of forming a pattern through the use of the photoresist composition according to claim 9.
